(19) United States
(12) Patent Application Publication
(10) Pub. No.: US 2007/0027120 A1
(43) Pub. Date: Feb. 1, 2007

(54) Methods of using vitamin D compounds in the treatment of myelodysplastic syndromes

(76) Inventors: Martha J. Whitehouse, San Francisco, CA (US); John G. Curd, Hillsborough, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/516,776

(22) Filed: Sep. 7, 2006

Related U.S. Application Data
(63) Continuation of application No. 10/841,820, filed on May 10, 2004.
Application No. 10/841,820 is a continuation-in-part of application No. 10/703,140, filed on Nov. 6, 2003, now abandoned.
(60) Provisional application No. 60/424,546, filed on Nov. 6, 2002.

(51) Int. Cl. A61K 31/59

(52) U.S. Cl. 514/167

(57) ABSTRACT

Methods of treating MDS, or ameliorating a symptom thereof, are disclosed. Specific methods encompass the administration of one or more vitamin D compounds, or a pharmaceutically acceptable salt, solvate, hydrate, stereoisomer, clathrate, or prodrug thereof, alone or in combination with one or more additional active agents. Other methods include intermittent administration of a high dose of one or more vitamin D compounds, or a pharmaceutically acceptable salt, solvate, hydrate, stereoisomer, clathrate, or prodrug thereof, alone or in combination with one or more additional active agents. Such intermittent administration allows high doses of the vitamin D compounds to be administered while minimizing or eliminating hypercalcemia.

### FIELD OF THE INVENTION

This invention relates, in part, to methods of treating myelodysplastic syndromes, or ameliorating one or more symptoms thereof, which comprise the administration of a vitamin D compound, or a pharmaceutically acceptable salt, solvate, hydrate, stereoisomer, clathrate, or prodrug thereof, as monotherapy or in combination with other therapeutic agents. The vitamin D compound can be administered in high doses to treat MDS, or ameliorate a symptom thereof, using intermittent administration to avoid side effects such as hypercalcemia.

### BACKGROUND OF THE INVENTION

### Pathobiology of MDS

Myelodysplastic syndrome (MDS) refers to a diverse group of hematopoietic stem cell disorders. MDS is characterized by a cellular marrow with impaired morphology and maturation (dysmyelopoiesis), peripheral blood cytopenias, and a variable risk of progression to acute leukemia, resulting from ineffective blood cell production. See The Merck Manual 953 (17^{th} ed. 1999) and List et al., 1990, J. Clin. Oncol. 8:1424-1441.

MDS is primarily a disease of elderly people, with the median onset in the seventh decade of life. The median age of these subjects is 65 years, with ages ranging from the early second decade of life to as old as 80 years or older. However, myelodysplasia may also afflict children, who present with similar clinical manifestations as adults. See Heany et al., 1999, New Eng. J. Med. 340:1649-60. Genetic abnormalities such as Down's syndrome are present in approximately 30% of children with MDS and are thought to predispose such children to myelodysplastic syndrome. See id.

MDS may be characterized as either primary or secondary, as subjects who survive malignancy treatment with certain chemotherapeutic agents or radiotherapy have a high incidence of developing secondary MDS or acute leukemia. See Zeidman et al., 1995, Haematologia (Budap) 27:23-8. About 60-70% of subjects do not have an obvious exposure or cause for MDS and are classified as primary MDS subjects. However, a nonspecific history of exposure to indeterminable chemicals or radiation 10-15 years prior to onset of disease may be present in some subjects. Exposure to compounds including, but not limited to, benzene, insecticides, weed killers and fungicides is correlated with increased incidence of MDS. See West et al., 2000, Blood 95:2093-7 and Goldberg et al., 1990, Cancer Res. 50:6876-81. Secondary MDS describes development of MDS or acute leukemia after exposure to genotoxic chemotherapy drugs or radiation during treatment for an unrelated malignancy. See Zeidman et al., 1995, Haematologia (Budap) 27:23-8. These drugs are associated with a high incidence of chromosomal abnormalities following exposure and at the time of MDS or acute leukemia diagnosis.

Further, MDS is associated with severe cytopenias and their attendant clinical complications. Possible manifestations of these cytopenias include increased risk of infection due to neutropenia and neutrophil dysfunction, bleeding due to thrombocytopenia and platelet dysfunction, and fatigue due to anemia. Other complications are development of myelofibrosis, which can accelerate decline in blood counts and increase transfusion requirements. See Heany et al., 1999, New Eng. J. Med. 340:1649-60 and Lambertenghi-Deliliers et al., 1992, Leuk. Lymphoma 8:51-5. Another major clinical issue for patients with MDS is the potential for the disease to evolve to acute myeloid leukemia (AML). Any or all of these manifestations can lead to shortened survival of afflicted subjects.

In MDS, an initial hematopoietic stem cell injury can be caused by, among other factors, cytotoxic chemotherapy, radiation, virus, chemical exposure and genetic predisposition. The early stages of MDS are primarily characterized by cytopenias, including anemia, neutropenia, and thrombocytopenia. The disease course varies in each subject, with some cases behaving as an indolent disease and others behaving aggressively with a very short clinical course that quickly converts into an acute leukemia.

An international group of hematologists, the French-American-British (FAB) Cooperative Group, classified MDS disorders into five subgroups, differentiating them from acute myeloid leukemia. See The Merck Manual 954 (17^{th} ed. 1999); Bennett et al., 1985, Ann. Intern. Med. 103: 620-625; and Besa, 1992 Med. Clin. North Am. 76(3): 599-617. According to the FAB classification, there are two subgroups of refractory anemia characterized by five percent or less myeloblasts in bone marrow: (1) refractory anemia (RA) and; (2) RA with ringed sideroblasts (RARS), defined morphologically as having 15% erythroid cells with abnormal ringed sideroblasts, reflecting an abnormal iron accumulation in the mitochondria. Besa, 1992 Med. Clin. North Am. 76(3): 599-617. There are also two subgroups of refractory anemias with greater than five percent myeloblasts: (1) RA with excess blasts (RAEB), defined as 6-20% myeloblasts, and (2) RAEB in transformation (RAEB-T), with 21-30% myeloblasts. Finally, the fifth and most difficult to classify type of MDS is called chronic myelomonocytic leukemia (CMML). This subtype can have any percentage of myeloblasts but presents with a monocytosis of 1000/dL or more. See id.; Harris et al., 1999, J. Clin. Oncol. 17:3835-49.

More recently, the World Health Organization has proposed a classification system for MDS called the International Prognostic Scoring System (IPSS). This system classifies MDS disorders into four prognostic categories on the basis of percentage of bone marrow blasts, cytogenetic subgroup, and number of cytopenias. See Greenberg et al., 1998, Blood 89:2079-88 and Bennett, 2000, Int. J. Hematol. 72:131-33. While the FAB classification system is still in use, the IPSS better predicts disease progression to acute myelogenous leukemia (AML) and patient survival. According to the IPSS, the values assigned to each classification variable as shown by table 1, below, are added together to determine the prognostic category of the MDS as shown by table 2, below. See Greenberg et al., 1998, Blood 89:2079-88. Median survival time for patients with low risk MDS is 5.7 years, while the median survival time for patients with high risk MDS is only 0.4 years. Median survival time for patients with intermediate-1 and -2 MDS is 3.5 and 1.2 years, respectively. See id.

TABLE 1

| | Survival and Evolution Score Value | | | | |
|---|---|---|---|---|---|
| Prognostic Variable | 0 | 0.5 | 1.0 | 1.5 | 2.0 |
| Marrow Blasts | <5 | 5-10 | — | 11-20 | 21-30 |
| (percentage) | | | | | |
| Karyotype | Good | Intermediate | Poor | | |
| Cytopenias (total | 0-1 | 2-3 | | | |

TABLE 2

| Risk Category | Combined Score |
|---|---|
| Low | 0 |
| Intermediate -1 | 0.5-1.0 |
| Intermediate -2 | 1.5-2.0 |
| High | ≧2.5 |

The actual incidence of MDS in the U.S. is unknown. MDS was first considered a distinct disease in 1976 and occurrence was then estimated at 1500 new cases every year. At that time, only subjects with less than five percent myeloblasts were considered to have this disorder. Recent (1999) statistics estimate 13,000 new cases per year and about 1000 cases per year in children, surpassing chronic lymphocytic leukemia as the most common form of leukemia in the western hemisphere. The perception that the incidence is increasing may be due to improvements in recognition and criteria for diagnosis. The disease is found worldwide.

### Existing MDS Treatments

The current therapies of MDS are based on the mechanisms predominating at a particular phase in the disease process. For younger subjects, bone marrow transplantation with a matched donor is the preferred treatment, but older subjects are often not candidates for such aggressive interventions since many are symptomatic from the anemia and are transfusion dependent. Hematopoietic growth factors or cytokines can be used to stimulate blood cell development and are effective in a subset of subjects. Other treatments include supportive care with transfusions of red cells and platelets combined with aggressive treatment of infections. In addition, many other classes of potentially therapeutic agents have also been assessed for efficacy in treating myelodysplastic syndrome, with limited success. Such classes include immunomodulators, cytotoxic agents, agents that affect RNA transcription, derivatives of vitamins A, E, and K, agents that specifically bind biological targets related to MDS, signal transduction inhibitors, cytoprotective agents, and arsenic-containing compounds.

Bone marrow transplantation has been used in subjects with poor prognosis or late-stage MDS. See Epstein et al., 1985 Surg. Ann. 17:23-29. Unfortunately, bone marrow transplantation is invasive, painful for both the donor and recipient, and can cause severe to fatal complications in the recipient. Standard allogeneic transplant treatments rely on maximally tolerated doses of chemotherapy and total body irradiation to eradicate disease and immunosuppress the recipient to allow engraftment and prevent graft rejection. Post transplant immunosuppression is used to induce tolerance and control graft versus host disease. Thus, allogeneic transplantations have been essentially limited to treatment of young, healthy subjects and must be administered in specialized inpatient units. Transplant related mortality is approximately 20-25% under the best conditions, and can be as high as 30-35%. See Deeg et al., 2000, Leuk. Res. 24:653-63. For this reason, very few transplants have been performed for subjects older than fifty years of age and have been limited to subjects with otherwise fatal diseases.

Repeated transfusions in subjects with symptomatic refractory anemia are associated with clinical risks of the transmission of infectious diseases, transfusion reactions and cardiovascular overload. In addition, multiple transfusions, such as about 20-30 transfusions, may cause secondary hemochromatosis, a condition that at least requires close monitoring of serum iron and often requires daily chelation therapy.

Hematopoietic growth factors or cytokines are an alternative approach to treating MDS and stimulating blood cell development. See Dexter, 1987 Cell Sci. 88:1-6; Moore, 1991 Annu. Rev. Immunol. 9:159-91; and Besa, 1992, Med. Clin. North Am. 76(3): 599-617. Hematopoietic growth factors are hormones involved in the process of blood cell formation. The treatment involves stimulating the proliferation of a small number of self-renewing stem cells that give rise to lineage-specific progenitor cells that subsequently proliferate and differentiate to produce mature circulating blood cells. See Metcalf, 1985, Science 229:16; Dexter, 1987, J. Cell Sci. 88:1-6; Golde et al., 1988, Scientific American: 62-71; Tabbara et al., 1991, Anti-Cancer Res. 11:81-90; Ogawa, 1989, Environ. Health Persp. 80:199-207; and Dexter, 1989, Med. Bull. 45:337-49. The most well-characterized growth factors include erythropoietin (EPO), granulocyte macrophage colony stimulating factor (GM-CSF) and granulocyte colony stimulating factor (G-CSF). In addition to inducing proliferation and differentiation of hematopoietic progenitor cells, such cytokines also activate a number of functions of mature blood cells, including influencing the migration of mature hematopoietic cells. See Stanley et al., 1976, J. Exp. Med. 143:631-47; Schrader et al., Proc. Natl. Acad. Sci. U.S.A., 1981, 78:323-7; Moore et al., 1980, J. Immunol. 125:1302-5; Kurland et al., Proc. Natl. Acad. Sci. U.S.A., 1979, 76:2326-30; Handman et al., 1979, J. Immunol. 122:1134-7; Vadas et al., 1983, Blood 61:1232; Vadas et al., 1983, J. Immunol. 130:795-9; and Weisbart et al., 1986, J. Immunol. 137:3584-87.

Recombinantly produced hematopoietic growth factors such as r-HuEPO (recombinant human erythropoietin; epoetin alfa; EPOGEN®, Amgen; PROCRIT®, Ortho Biotech) and r-metHuG-CSF (recombinant human granulocyte colony-stimulating factor; filgrastim; NEUPOGEN®, Amgen) have been effective at supporting red blood cell (RBC) and neutrophil production, respectively, in a subset of subjects. See Hellström-Lindberg, et al., 1998, Blood 92:68-75 and Hellström-Lindberg, et al., 1997, Br. J. Haematol. 99:344-51. Concomitantly increased hemoglobin levels have resulted in improvements in the quality of life in several large community-based trials in cancer subjects. See, e.g., Glaspy et al., 1997, J. Clin. Oncol. 15:1218-34 and Detetri et al, 1998, J. Clin. Oncol. 16:3412-25.

However, the anemia of MDS is often serious and refractory to hematopoietic growth factors or cytokines. Anemia may aggravate conditions common to elderly subjects, including but not limited to congestive heart failure, coronary artery disease and chronic lung disease. Only about 20% of subjects respond to EPO alone, and about 40% of subjects respond to EPO administered with G-CSF. See Hellström-Lindberg et al., 1998, Blood 92:68-75 and Hellström-Lindberg et al., 1997, Br. J. Haematol. 99:344-51. A serum erythropoietin level of <200 mU/mL is often predictive of a response to EPO, but responsiveness depends upon the stage of the disease with rates of 21% in refractory anemia and refractory anemia with ring sideroblasts, but only 8% in refractory anemia with excess blasts. See Hellström-Lindberg et al., 1997, Br. J. Haematol. 99:344-51 and Hellström-Lindberg, 1995, Br. J. Haematol. 89:67-71. Thus, treatment with EPO, G-CSF, or other growth factors is not effective to treat all, or even most, subjects with MDS.

Other growth factors that have been administered in the treatment of MDS include thrombopoietin, interferon-α, interleukin-1, interleukin-2, interleukin-3, interleukin-6, interleukin-8, interleukin-11, and interleukin-12. While many of these factors show promise in vitro and in preclinical studies, clinical trials to date have met with little to no success. See Schipperus et al., 1991 Br. J. Haematol. 77:515-22; Ganser et al., 2000, Ann. Hematol. 79:30-5; Musto et al., 2001, Haematologica 86:44-51; Gordon, Semin. Hematol. 1999, 36(4 Suppl 6):21-4; Zwierzina et al., 1993, Scand. J. Immunol. 37:322-8; Estey et al., 2002, Blood 99:4343-9; Pan et al., 2000, Leukemia 14:1634:41; Hofmann et al., 1999, Eur. J. Haematol. 62:336-40; Hofmann et al., 1999, Ann. Hematol. 78:125-30; Haznedaroglu et al., 2002, Clin. Appl. Thromb. Hemost. 8:193-212; and Ogata et al., 2000, Int. J. Hematol. 72:173-7.

Attempts have also been made to treat MDS with immunomodulators, cytotoxic agents, agents that affect RNA transcription, derivatives of vitamins A, E, and K, agents that specifically bind biological targets related to MDS, signal transduction inhibitors, cytoprotective agents, and arsenic-containing compounds. For example, immunomodulators that have been tested as possible therapeutic agents for MDS include anti-thymocyte globulin (ATG), anti-lymphocyte globulin (ALG), thalidomide, prednisone, cyclosporin A (CyA), dexamethasone, and pentoxifylline. See, e.g., Molldrem et al., 2002, Ann. Intern. Med. 137:156; Rong et al., 2002, Eur. J. Haematol. 68:210; Tsirigotis et al., 2002, Leuk. Res. 26:965; Hisconmex et al., 2001, Leuk. Lymphoma 42:665; Ohga et al., 2002, Br. J. Haematol. 118:313; Greipp, 2000, Curr. Treat. Options Oncol. 1:119-26; and Raza et al., 2000, Hematol 5:274-84. Tested cytotoxic agents include cytarabine, melphalan, topotecan, fludarabine, etoposide, idarubicin, daunorubicin, mitoxantrone, cisplatin, paclitaxel, and cyclophosphamide. See, e.g., Garcia-Manero et al., 2002, Haematologica 87:804; Beran et al., 2001, Cancer 92:1999; Sackmann-Muriel et al., 1996, Leuk. Res. 20:973; Oosterveld et al., 2002, Leukemia 16:1615; Hisconmex et al., 2001, Leuk. Lymphoma 42:665; Denzlinger et al., 2000, Br. J. Haematol. 108:93; Oosterveld et al., 2002, Leukemia 16:1615; and Lee et al., 2002, Am. J. Hematol. 68:237.

In addition, agents that affect RNA transcription that have been tested as potential therapies for MDS include decitabine, 5-azacytidine, depsipeptides, and phenylbutyrate. See, e.g., Daskalakis et al., 2002, Blood 100:2957; Gryn et al., 2002, Leuk. Res. 26:893; Ballard et al., 2002, Curr. Med. Chem. 9:471; Imanishi et al., 2002, J. Clin. Endocrinol. Metab. 87:4821; Silverman et al., 2002, J. Clin. Oncol. 20:2429; and Gore et al., 2002, Clin. Cancer Res., 8:963-970. Derivatives of vitamins A, E, and K that have been assessed as therapies for MDS include all trans retinoic acid, 13-cis-retinoic acid, tocopherol, and menatetrenone. See, e.g., Stasi et al., 2002, Blood 99:1578; Hofmann et al., 2000, Leukemia 14:1583; Takami et al., 2002, Ann. Hematol. 81:16; and Besa et al, 1998, Leuk. Res. 22:741. Agents that specifically bind biological targets related to MDS that have been tested as potential therapies include anti-VEGF, gemtuzumab ozogamicin, and TNFR:Fc. See, e.g., Verstovsek et al., 2002, Br. J. Haematol. 118:151; List, 2002, Oncologist 7 Suppl 1:39; and Rosenfeld et al., 2002, Leuk. Res., 26:721. Signal transduction inhibitors that have been tried as therapeutic agents for MDS include farnesyl transferase inhibitors such as ZARNESTRA™ and SARASAR™ and tyrosine kinase inhibitors such as SU5416, SU6668, and PTK787/ZK222584. See, e.g., Kurzrock, 2002, Semin. Hematol., 39(3 Suppl 2):18; Cortes et al., 2002, Semin. Hematol 39(3 Suppl 2):26; List, Oncologist 7 Suppl 1:39 (2002); and Cheson et al., 2000, Semin. Oncol. 27:560. Finally, the cytoprotective agent and arsenic-containing compound that have been assessed as potential therapies for MDS are amifostine and arsenic trioxide, respectively. See, e.g., Arboscello et al., 2002, AntiCancer Res. 22:1819; Invernizzi et al., 2002, Br. J. Hematol. 118:246; and Miller, 2002, Oncologist 7 Suppl 1:14. With one exception, none of these treatments has unambiguously resulted in significant therapeutic effect in subjects with MDS.

The single exception to the otherwise uniformly mediocre to poor results observed in testing the above described compounds as potential therapies for MDS is 5-azacytidine. Silverman reported that some 60% of patients who were injected subcutaneously with 75 mg/m²/day of 5-azacytidine for 7 days out of 28 experienced at least a partial response, with 7% of the patients enjoying a complete response. See Silverman et al., 2002, J. Clin. Oncol. 20:2429. However, this therapeutic regimen suffers from several drawbacks. 5-Azacytidine is exceedingly toxic and can cause severe nausea and emesis in the subjects to whom it is administered. Also, the method of administration of this protocol is inconvenient for patients who must visit the administering clinic daily for the week of treatment. Finally, administration of 5-azacytidine initially causes the cytopenias of subjects with MDS to worsen before they later improve, which can be dangerous or lethal to some patients. Therefore, there remains a need for safe and effective methods of treating and managing MDS. Particularly, a method that is effective at treating the anemia associated with MDS and reducing the RBC transfusion requirements would be of clinical benefit.

### Vitamin D Compounds

Vitamin D is a generic term for a family of secosteroids that have affinity for the vitamin D receptor, and are involved in the physiologic regulation of calcium and phosphate metabolism. See Harrison's Principles of Internal Medicine: Part Eleven, “Disorders of Bone and Mineral Metabolism,” E. Braunwald et al., (eds.), 1987, McGraw-Hill, New York at Chapter 335, pp. 1860-1865, Stumpf et al., 1979, Science 206:1188-90, and Holick, 1995, Bone 17:107S-11S. Vitamin D exhibits a complex set of actions and mechanisms of synthesis. Cholecalciferol (vitamin D₃) is synthesized in the skin following ultraviolet radiation from 7-dehydrocholesterol. Vitamin D₂, an analog of vitamin D₃, can be ingested from the diet. Two sequential hydroxylations of vitamin D₂ are necessary for full biological activity. The first hydroxylation, which takes place in the liver, results in the formation of 25-hydroxycholecalciferol, while the second hydroxylation takes place in the kidney and results in the formation of the most potent biological metabolite of vitamin D: 1α,25-dihydroxycholecalciferol (also known as calcitriol).

Calcitriol maintains calcium homeostasis by modulating intestinal absorption, urinary excretion, and mobilization from skeletal bone. These effects can be exerted through both genomic and non-genomic pathways. The genomic responses are mediated by calcitriol binding the nuclear vitamin D receptor (VDR). The VDR is a ligand-activated transcription factor that activates transcription of genes regulated by the vitamin D response element within their promoter/enhancer regions. See Mangelsdorf et al., 1995, Cell 83:835-9. The non-genomic pathways are mediated by an as-yet uncharacterized membrane-bound receptor.

In addition, the VDR has been found in cells from diverse organs not involved in calcium homeostasis. See Miller et al., 1992, Cancer Res. 52:515-520. In addition to influencing calcium homeostasis, vitamin D compounds have been implicated in osteogenesis, modulation of the immune response, modulation of insulin secretion by pancreatic B cells, muscle cell function, and differentiation and growth of epidermal and hematopoietic tissues.

Attempts have been made to use vitamin D compounds in the treatment of cancer. For example, certain vitamin D compounds and analogs possess potent anti-leukemic activity by virtue of their ability to induce differentiation of leukemic cells to non-malignant macrophages (monocytes) and are therefore useful in the treatment of leukemia. See Suda et al., U.S. Pat. No. 4,391,802; Partridge et al., U.S. Pat. No. 4,594,340. Antiproliferative and differentiating actions of calcitriol and other vitamin D₃ analogs have also been reported with respect to the treatment of prostate cancer. See Bishop et al., U.S. Pat. No. 5,795,882. Vitamin D compounds have also been implicated in the treatment of skin cancer (See Chida et al., 1985, Cancer Res. 45:5426-5430), colon cancer (See Disman et al., 1987, Cancer Res. 47:21-25), and lung cancer (See Sato et al., 1982, J. Exp. Med. 138:445-446). Other reports suggesting important therapeutic uses of vitamin D compounds are summarized in Rodriguez et al., U.S. Pat. No. 6,034,079.

Vitamin D compounds have also been administered in combination with other pharmaceutical agents, in particular cytotoxic agents, for the treatment of hyperproliferative disease. For example, it has been shown that pretreatment of hyperproliferative cells with Vitamin D compounds followed by treatment with cytotoxic agents enhances the efficacy of the cytotoxic agents (U.S. Pat. Nos. 6,087,350 and 6,559,139).

Although the administration of vitamin D compounds may result in substantial therapeutic benefits, their use as a treatment for cancer or MDS has been severely limited by the effects that these compounds have on calcium metabolism. At the levels required for effective use in vivo, vitamin D compounds can induce markedly elevated and potentially dangerous blood calcium levels by virtue of their inherent calcemic activity. That is, the clinical use of calcitriol and other vitamin D compounds to treat cancer or MDS has been precluded, or severely limited, by the risk of hypercalcemia.

It has been shown that the problem of systemic hypercalcemia can be overcome by “high dose pulse administration” (HDPA) of a sufficient dose of an active vitamin D compound such that an anti-proliferative effect is observed while avoiding the development of severe hypercalcemia. According to U.S. Pat. No. 6,521,608, the active vitamin D compound may be administered no more than every three days, for example, once a week at a dose of at least 0.12 μg/kg per day (8.4 μg in a 70 kg person). Pharmaceutical compositions used in the HDPA regimen of U.S. Pat. No. 6,521,608 comprise 5-100 μg of active vitamin D compound and may be administered in the form for oral, intravenous, intramuscular, topical, transdermal, sublingual, intranasal, intratumoral, or other preparations. In a Phase I trial of weekly administration of calcitriol to patients with refractory malignancies, HDPA of calcitriol was shown to produce no dose-limiting toxicity and to produce mean peak calcitriol levels within the therapeutic range. Beer et al., Cancer 91:2431-39 (2001).

### Administration of Vitamin D Compounds in the Treatment of MDS

Although no pre-clinical animal model for MDS is available, calcitriol has been reported to alter the growth of marrow progenitors in vitro and promote development of monocytic progenitors in a dose-dependent manner. See Swanson et al., 1986, Blood 67:1154-1161. Furthermore, evidence suggests that a local deficiency of calcitriol may exist in the bone marrow microenvironment in some MDS subjects. See Blazsek et al., 1996, Cancer Detect. Prevent. 20:31-42. Calcitriol appears to improve the anemia and reduce the need for erythropoietic agents in subjects on hemodialysis. See Goicoechea et al., 1998, Nephron 78:23-27. While the preclinical data regarding use of calcitriol to treat MDS are consistently positive, results from clinical trials of calcitriol as a therapeutic agent have been limited in scope and mediocre in response. See Morosetti et al., 1996, Semin. Hematol. 33:236-245. One reason for these results has been induction of hypercalcemia with the administration of only 2 μg/day of calcitriol. See Koeffler et al., 1985, Cancer Treat. Rep. 69:1399-1407. A maximum of 0.75 μg/day of calcitriol has been administered to subjects with MDS without hypercalcemia. See Mellibovsky et al., 1998, Brit. J. Haemotol. 100:516-520. These studies report that the administered doses of vitamin D compounds provide some benefit to some subjects with some forms of MDS, but the doses could not be increased to more effective levels without causing hypercalcemia in the subjects.

As the foregoing review of past and present therapies for MDS demonstrates, a large number of potential therapeutic agents have been and are being tested for their ability to treat MDS. Nonetheless, none of these agents has met with unequivocal success in clinical assessments. Indeed, there is still no Food and Drug Administration-approved agent with an indication for this disease. See List, 2002, Oncologist 7(suppl 1):39-49. Furthermore, many of the more aggressive therapies, such as bone marrow transplant and high-dose chemotherapy, are inappropriate for a large percentage of subjects with MDS because of their advanced age and weakened condition. See id. Accordingly, there remains an unmet need for safe and effective methods of treating MDS, or ameliorating a symptom thereof. Particularly, a method that is effective at treating the anemia associated with MDS and reducing transfusion requirements would be of clinical benefit. Calcitriol and other vitamin D compounds have been shown to exert such a clinical benefit, but their usefulness as a therapeutic agent has been limited by induction of hypercalcemia. Therefore, methods of treating MDS while not causing unwanted and dangerous side effects such as hypercalcemia are required.

Citation or discussion of a reference herein shall not be construed as an admission that such is prior art to the present invention.

### SUMMARY OF THE INVENTION

This invention encompasses methods and compositions for the treatment of myelodysplastic syndrome (MDS), or ameliorating a symptom thereof, particularly the anemia of MDS, comprising administering to a subject in need thereof a therapeutically effective dose of vitamin D compounds, or pharmaceutically acceptable salts, solvates, hydrates, stereoisomers, clathrates, or prodrugs thereof, while avoiding or minimizing hypercalcemia. These methods and compositions can be used for the treatment of MDS, or amelioration of a symptom thereof, with few or no associated symptoms of hypercalcemia.

In some aspects, the methods of the invention comprise administering intermittently a therapeutically effective dose of a vitamin D compound and optionally administering one or more additional active agents. The dose of the vitamin D compound can be a high dose, as intermittent administration of the vitamin D compounds according to the methods of the invention allows a high dose to be administered to a subject without causing hypercalcemia. The vitamin D compound can be any vitamin D compound without limitation. In preferred embodiments, the vitamin D compound is an active vitamin D compound such as calcitriol. A therapeutically effective dose of a vitamin D compound can be a dose between about 3 μg/day to about 300 μg/day, or any range of doses therein as described below. In certain embodiments, the vitamin D compounds can be administered not more than once every three days. In preferred embodiments, the vitamin D compound is administered about once per week. A therapeutically effective dose of an active vitamin D compound is preferably between about 3 μg/day to about 300 μg/day, more preferably between about 5 μg/day to about 200 μg/day, more preferably between about 15 μg/day to about 105 μg/day, more preferably between about 15 μg/day to about 90 μg/day, more preferably between about 20 μg/day to about 80 μg/day, more preferably between about 35 μg/day to about 75 μg/day, more preferably between about 30 μg/day to about 60 μg/day, and even more preferably about 45 μg. In certain embodiments, the therapeutically effective dose of a vitamin D compound safely achieves peak plasma concentrations of the vitamin D compound of at least about 0.5 nM, more preferably about 1-7 nM, and even more preferably about 3-5 nM. While any vitamin D compound may be used according to the methods of the invention, preferred vitamin D compounds achieve peak plasma concentrations rapidly and are eliminated quickly.

In further embodiments, the invention provides methods for the treatment of MDS, or amelioration of a symptom thereof, comprising administering a therapeutically effective dose of a vitamin D compound in combination with one or more additional active agents. The therapeutically effective dose of the vitamin D compound can be any dose, in combination with the one or more additional active agents, effective to treat MDS or ameliorate a symptom thereof. In certain embodiments, the therapeutically effective dose of the vitamin D compound is a high dose. The additional active agents can be one or more growth factors, e.g., hematopoietic growth factors or cytokines; immunomodulators; cytotoxic agents, e.g., antimetabolites, anti-microtubule agents, alkylating agents, platinum agents, anthracyclines, antibiotic agents, or topoisomerase inhibitors; agents that affect RNA transcription; derivatives of vitamins A, E, and K; agents that specifically bind biological targets related to MDS; signal transduction inhibitors; cytoprotective agents; or arsenic-containing compounds. Examples of the hematopoietic growth factors or cytokines include, but are not limited to, erythropoietin (EPO) and granulocyte colony stimulating factor (G-CSF), and more particularly recombinant human erythropoietin (r-HuEPO), and recombinant methionyl human granulocyte colony stimulating factor (r-metHuG-CSF). In further embodiments, the present invention provides pharmaceutical compositions comprising one or more vitamin D compounds and one or more additional active agents.

In the methods of the present invention, a vitamin D compound and optionally one or more additional active agents can be administered in the form of a pharmaceutical composition, a single unit dosage form, or article of manufacture suitable for use in treating MDS, or ameliorating a symptom thereof, which comprises one or more vitamin D compounds, or pharmaceutically acceptable salts, solvates, hydrates, stereoisomers, clathrates, or prodrugs thereof. The vitamin D compound(s) and optionally one or more additional active agents can be formulated in any pharmaceutical composition known to those of skill in the art. In certain embodiments, the vitamin D compounds are administered in oral or intravenous formulations. Preferred oral formulations include emulsion pre-concentrates which comprise one or more vitamin D compounds, a lipophilic phase component, and a surfactant. In certain embodiments of the invention, the compositions for the treatment of MDS, or amelioration of a symptom thereof, comprise a therapeutically effective dose of a vitamin D compound, or a pharmaceutically acceptable salt, solvate, hydrate, stereoisomer, clathrate, or prodrug thereof in combination with one or more additional active agents.

The methods and compositions of the present invention are useful for the treatment of MDS, or amelioration of a symptom thereof, in a subject, preferably a human subject. Significantly, the methods and compositions of the present invention can be used for the treatment of MDS, or amelioration of a symptom thereof, with active vitamin D compounds such as calcitriol, while minimizing or avoiding the effects of hypercalcemia.

### BRIEF DESCRIPTION OF THE FIGURES

FIG. 1 provides plasma concentrations of calcitriol as a function of time.

FIGS. 2A-2C provide hemoglobin concentration (in grams per deciliter) and red blood cell transfusion frequency (in units transfused) as a function of time for patient #1 (FIG. 2A), patient #2 (FIG. 2B), and patient #3 (FIG. 2C).

### DETAILED DESCRIPTION OF THE INVENTION

The present invention provides methods and compositions for treating myelodysplastic syndrome, or ameliorating a symptom thereof, with one or more vitamin D compounds, preferably an active vitamin D compound such as calcitriol, while minimizing or eliminating the risks of hypercalcemia. In certain aspects of this invention, the one or more vitamin D compounds are administered in combination with one or more additional active agents.

### Definitions

“Active vitamin D compound” refers to a vitamin D compound that is biologically active when administered to a subject or contacted with cells. The biological activity of a vitamin D compound can be assessed by assays described herein or well-known to one of skill in the art such as, e.g., immunoassays (e.g., enzyme-linked immunoassays (“ELISAs”)) that measure the expression of a gene. Vitamin D compounds exist in several forms with several different levels of activity in the body. For example, a vitamin D compound may be partially activated by first undergoing hydroxylation in the liver to 25-hydroxycholecalciferol and then may be fully activated in the kidney to 1α,25-dihydroxycholecalciferol, which is also known as, inter alia, “calcitriol.” Calcitriol, however, is the principal biologically active form of vitamin D in humans and does not require further modification in the body for immediate utilization.

“Calcemic index” refers to a measure of the relative ability of a drug to generate a calcemic response. Examples of such measurement are demonstrated in Bouillon et al., 1995, Endocrine Reviews 16:200-7. A calcemic index of 1 corresponds to the relative calcemic activity of calcitriol. A calcemic index of about 0.01 corresponds to the calcemic activity of calcipotriol. A calcemic index of 0.5 would correspond to a drug having approximately half the calcemic activity of calcitriol. The calcemic index of a drug can vary depending on the assay used, e.g., whether measuring stimulation of intestinal calcium absorption (ICA) or bone calcium mobilizing activity (BCM), as reported in Hurwitz et al., 1967, J. Nutr. 91:319-323 and Yamada et al., 1988, Mol. Cell. Endocrinol. 59:57-66. Relative calcemic activity is best expressed in relation to the calcemic activity of calcitriol, which is one of the best characterized vitamin D compounds.

“Clinical hypercalcemia” refers to one or more of the signs or symptoms of hypercalcemia. Early manifestations of hypercalcemia include weakness, headache, somnolence, nausea, vomiting, dry mouth, constipation, muscle pain, bone pain, or metallic taste. Late manifestations include polydipsia, polyuria, weight loss, pancreatitis, photophobia, pruritus, renal dysfunction, aminotransferase elevation, hypertension, cardiac arrhythmias, psychosis, stupor, coma and ectopic calcification. Hypercalcemia can be life-threatening and is thus typically to be avoided in vitamin D compound administration

“Emulsion pre-concentrate” refers to a formulation capable of providing an emulsion upon contact with a polar medium such as water. The term “emulsion” refers to a colloidal dispersion comprising a polar medium such as water and organic components including but not limited to hydrophobic, i.e. lipophilic, organic components and encompasses both conventional emulsions and sub-micron droplet emulsions. The term “sub-micron droplet emulsion,” refers to an emulsion wherein the droplets or particles forming the colloidal dispersion of organic components have an average maximum dimension of less than about 1000 nm.

“Hypercalcemia” refers to a condition in which the blood calcium concentration is greater than normal (although the normal value can vary slightly depending on the measuring technique used). Although the concentration that is considered “normal” will vary slightly with variation in measurement techniques, a value above 10.5 mg/dL in humans is considered hypercalcemia. Hypercalcemia can be divided into grades 0-4. Grade 0 corresponds to a value of blood calcium concentration that is less than 10.6 mg/dL; Grade 1 corresponds to a value of blood calcium concentration of 10.6-11.5 mg/dL; Grade 2 corresponds to a value of blood calcium concentration of 11.6-12.5 mg/dL; Grade 3 corresponds to a value of blood calcium concentration of 12.6-13.5 mg/dL; and Grade 4 corresponds to a value of blood calcium concentration that is greater than 13.5 mg/dL. See, e.g., U.S. Pat. No. 6,521,608.

“In combination” refers to the use of more than one therapeutic agent. The use of the term “in combination” does not restrict the order in which therapeutic agents are administered to a subject with MDS. A first therapeutic agent can be administered prior to, concurrently with, after, or within any cycling regimen involving the administration of a second therapeutic agent to a subject with MDS. For example, the first therapeutic agent can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before a second therapeutic agent; or the first therapeutic agent can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after a second therapeutic agent.

“Isomer” refers to a chemical compound having the same chemical formula as another but different structure. An example of a “constitutional isomer” of propanol is isopropanol, wherein the compounds have the same molecular formula but differ in the placement of the connections between the atoms. An example of a “stereoisomer” is an “enantiomer,” which is any compound that is the mirror image of another compound. Another example of a stereoisomer is a diastereomer, which is any stereoisomer that contains more than one chiral center but is not an enantiomer.

“Intermittent” administration refers to a method of achieving periodically high blood concentrations of vitamin D compounds without the onset of hypercalcemia. The method of intermittent administration comprises periodically dosing a subject with a high level of one or more vitamin D compounds. Intermittent administration may comprise, for example, but not by way of limitation, administering the one or more vitamin D compounds not more than every three days, about once every four days, about once every five days, about once every six days, about once per week, about once every nine days, about once every two weeks, about once every three weeks, or about once every four weeks. The period of intermittent administration may continue for one, two, three, or four weeks, or one, two, three, four, five, or six months, or longer. The intermittent dosing schedules can comprise administration of vitamin D compounds that is more or less frequent than those mentioned thus far, or that continues for longer or shorter treatment periods, depending on the pharmacokinetics and pharmacodynamics of the pharmaceutical agent employed. One of skill in the art will readily understand the potential need to adjust the periodic dosing regimens, and that any periodic dosing schedule that includes the administration of high doses of vitamin D compounds without the onset of hypercalcemia is within the scope of the invention. An example of a dosing schedule that can be used by the methods of the present invention is provided in U.S. Pat. No. 6,521,608, which is incorporated by reference.

“Metabolite” refers to a substance that results after the body has processed, i.e. metabolized, another substance. An example of a series of metabolites may begin with 1,25-dihydroxyergocalciferol, the most active form of vitamin D₂, which is a metabolite of 25-hydroxyergocalciferol, which is a metabolite of ergocalciferol (vitamin D₂), which is a metabolite of ergosterol. Another example of a series of metabolites may begin with 1,25-dihydroxycholecalciferol (calcitriol), which is a metabolite of 25-hydroxycholecalciferol, which is a metabolite of cholecalciferol (vitamin D3), which is a metabolite of 7-dehydrocholesterol. Another example of a series of metabolites may begin with tachysterol, which is a metabolite of dihydrotachysterol, which is a metabolite of 25-hydroxydihydrotachysterol.

“Non-hypercalcemic vitamin D compound” refers to a vitamin D compound that has less of a tendency to produce the onset of hypercalcemia than a comparable dosage of calcitriol as assessed by assays well-known to one of skill in the art. Examples of such non-hypercalcemic vitamin D compounds include analogs of calcitriol such as Ro23-7553 and Ro24-5531 (1α,25-dihydroxy-16-ene-23-yne-26,27-hexafluorocholecalciferol) available from Hoffmann-LaRoche. Other examples of non-hypercalcemic vitamin D compounds can be found in U.S. Pat. No. 4,717,721, which is incorporated by reference herein in its entirety.

“Pharmaceutical formulation” refers to a composition comprising ingredients that are pharmaceutically acceptable for their intended use.

“Pharmaceutical agent” refers to one or more vitamin D compounds or one or more vitamin D compounds in combination with one or more active ingredients that are not vitamin D compounds, including but not limited to, bisphosphonates. The pharmaceutical agent can be administered in combination with other active ingredients as well, such as, for example, the administration of vitamin D compounds in combination with hematopoietic growth factors or cytokines in the treatment of MDS.

“Precursor” refers to a compound that can be transformed into another compound that is biologically active. An example of a series of precursors may begin with ergosterol, which is the precursor to ergocalciferol (vitamin D₂), which is the precursor to 25-hydroxyergocalciferol, which is the precursor to 1,25-dihydroxyergocalciferol, the most active form of vitamin D₂. Another example of a series of precursors may begin with 7-dehydrocholesterol, which is the precursor to cholecalciferol (vitamin D3), which is the precursor to 25-hydroxycholecalciferol, which is the precursor to 1,25-dihydroxycholecalciferol (calcitriol). Another example of a series of precursors may begin with tachysterol, which is the precursor to dihydrotachysterol, which is the precursor to 25-hydroxydihydrotachysterol.

“Refractory” and “non-responsive” refer to subjects treated with a currently available therapeutic agent for MDS which is not clinically adequate to relieve one or more symptoms associated with the MDS. Typically, such subjects suffer from severe, persistently active disease and require additional therapy to ameliorate the symptoms associated with their MDS.

“Synergistic” refers to a combination of therapeutic agents which is more effective than the additive effects of any two or more single agents. A synergistic effect of a combination of therapeutic agents permits the use of lower dosages of one or more of the agents and/or less frequent administration of said agents to a subject with MDS. The ability to utilize lower dosages of therapeutic agents and/or to administer said agents less frequently reduces the toxicity associated with the administration of said agents without reducing the efficacy of said agents in the treatment of MDS, or amelioration of a symptom thereof. In addition, a synergistic effect can result in improved efficacy of agents in the treatment of MDS, or amelioration of a symptom thereof. Finally, the synergistic effect of a combination of therapeutic agents may avoid or reduce adverse or unwanted side effects associated with the use of any single therapy.

“Subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey, such as a cynomolgus monkey, and a human), and more preferably a human.

“Therapeutic agents” refer to any agent(s) which can be used in the prevention or treatment of MDS, or amelioration of a symptom thereof. In certain embodiments, the term “therapeutic agents” refers to one or more vitamin D compounds. In other embodiments, the term “therapeutic agents” does not refer to a vitamin D compound. Preferably, a therapeutic agent is known to be useful, or has been or is currently being used, to prevent or impede the development, onset or progression of MDS, or ameliorate the symptoms of MDS.

A “therapeutically effective dose” refers to a dose of an ingredient that can achieve the desired therapeutic or prophylactic effects, such as, for example, a dose that can achieve a blood level of a vitamin D compound that is above normal for a sufficient period of time to have therapeutic benefit without clinically relevant toxicity. According to the methods of the invention, a therapeutically effective dose of vitamin D compounds can range from about 3 μg to about 300 μg, or any range of amounts therein. Higher peak blood levels of vitamin D compounds are associated with increased efficacy but at some point the benefit may be limited by toxicity. Specific regimens of administration allow higher doses to be administered safely, that is, without the onset of symptoms associated with hypercalcemia. In a specific embodiment, a therapeutically effective amount of a vitamin D compound (preferably an active vitamin D compound or a non-hypercalcemic vitamin D compound) is 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 μg or more. In certain embodiments, a therapeutically effective dose of an active vitamin D compound is preferably between about 3 μg/day to about 300 μg/day, more preferably between about 5 μg/day to about 200 μg/day, more preferably between about 15 μg/day to about 105 μg/day, more preferably between about 15 μg/day to about 90 μg/day, more preferably between about 20 μg/day to about 80 μg/day, more preferably between about 35 μg/day to about 75 μg/day, more preferably between about 30 μg/day to about 60 μg/day, and even more preferably about 45 μg. In certain embodiments, the therapeutically effective dose of vitamin D compound safely achieves peak plasma concentrations of the vitamin D compound of at least about 0.5 nM, more preferably about 1-7 nM, and even more preferably about 3-5 nM.

“Treat,” “treatment” and “treating” refer to administration of one or more prophylactic or therapeutic agents either before or after the onset of symptoms of MDS. “Treat,” “treatment” and “treating” further include “managing” MDS, which includes lengthening the time a subject remains in remission and/or preventing the reoccurrence of MDS in subjects at risk of suffering MDS. “Treat,” “treatment” and “treating” further include preventing the recurrence or onset of one or more symptoms of MDS in a subject. The symptoms associated with MDS include but are not limited to anemia, thrombocytopenia, neutropenia, bicytopenia (two deficient cell lines), and pancytopenia (three deficient cell lines).

A “vitamin D compound” refers to any form of chemical compound with an affinity for the vitamin D receptor (VDR). The vitamin D compounds of the present invention can concentrate in the blood to a therapeutically effective level. The VDR is a ligand-activated transcription factor, or intracellular receptor, which initiates transcription by binding to the vitamin D response element within the promoter/enhancer region of target genes. Examples of vitamin D compounds within the scope of the invention include but are not limited to calcitriol, 1,25-dihydroxyergocalciferol, calcifediol, 25-hydroxyergocalciferol, ergocalciferol, cholecalciferol, doxercalciferol, dihydrotachysterol, paracalcitol, as well as the derivatives, analogs, homologs, precursors and metabolites thereof. Preferred vitamin D compounds are active vitamin D compounds and include but are not limited to calcitriol and all of its derivatives, analogs, homologs, precursors and metabolites. The most preferred vitamin D compound is calcitriol.

### Vitamin D Compounds

In the methods of the present invention, the vitamin D compound can be any compound that binds to a vitamin D receptor and thus can be any vitamin D compound known to one of skill in the art. For instance, the term “vitamin D” traditionally refers to ergocalciferol (vitamin D₂) and cholecalciferol (vitamin D3), but the present invention encompasses the use of any vitamin D compound or its derivatives, analogs, homologs, precursors and metabolites. As such, the term vitamin D compound not only includes, for example, naturally occurring ergocalciferol and cholecalciferol, but also includes their respective precursors ergosterol and 7-dehydrocholesterol. Furthermore, the term vitamin D compound also includes the activated forms or metabolites of ergocalciferol and cholecalciferol, which include 25-hydroxyergocalciferol and 25-hydroxycholecalciferol (calcifediol) in addition to the most active forms, which are 1,25-dihydroxyergocalciferol and 1,25-dihydroxycholecalciferol (calcitriol). The chemical structure of calcitriol is as follows:

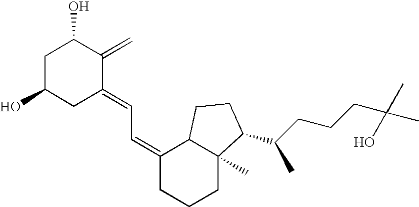

The vitamin D compound can be isolated from natural sources or synthesized by methods known to those of skill in the art. An example of a synthetic vitamin D analog is dihydrotachysterol. Dihydrotachysterol is a synthetic reduction product of tachysterol. Tachysterol is a byproduct formed during the irradiation of 7-dehydrocholesterol, the precursor to vitamin D₃. Dihydrotachysterol is ten times more active than its precursor tachysterol and is activated in the liver to the even more active 25-hydroxydihydrotachysterol. Other examples of synthetic vitamin D analogs are paricalcitol and doxercalciferol, which can be used to lower parathyroid hormone levels. Another example of a synthetic vitamin D analog is alfacalcidol, which is currently in clinical use in Canada for the treatment and prevention of renal bone disease, rickets, hypoparathyroidism and osteoporosis.

Active Vitamin D Compounds

The vitamin D compounds used in the present invention comprise active vitamin D compounds. While not intending to be bound by any particular theory or mechanism of action, vitamin D compounds can become activated, for example, through (1) ultraviolet conversion of 7-dehydrocholesterol in the skin to vitamin D₃ (cholecalciferol) and (2) dietary intake of either vitamin D₂ (ergocalciferol) or vitamin D₃. Both vitamin D₂ and vitamin D₃ compounds, for example, become fully active on target tissues when metabolically activated in the liver and kidney. Regardless of whether the vitamin D compound was a product of ultraviolet conversion in the skin or dietary intake, the next step in activation can be the introduction of a hydroxyl group in the side chain at the C-25 position by a hepatic enzyme known as CYP 27 (a vitamin D-25-hydroxylase). At this point, the partially activated vitamin D₂ and D₃ compounds are known as, inter alia, 25-hydroxyergocalciferol and 25-hydroxycholecalciferol, respectively. These partially activated compounds become fully activated in the mitochondria of kidney tissue by renal 25-hydroxyvitamin D-1-α-hydroxylase to produce 1α,25-(OH)2D₂, the primary biologically active form of vitamin D₂, and 1α,25-(OH)₂D₃ (calcitriol), the most biologically active form of vitamin D₃. The active vitamin D compounds of the present invention include but are not limited to the analogs, homologs and derivatives of vitamin D compounds described in the following patents, each of which is incorporated by reference herein in its entirety: U.S. Pat. No. 4,391,802 (1α-hydroxy vitamin D derivatives); U.S. Pat. No. 4,717,721 (α-hydroxy derivatives with a 17-side chain greater in length than the cholesterol or ergosterol side chains); U.S. Pat. No. 4,851,401 (cyclopentano-vitamin D analogs); U.S. Pat. No. 5,145,846 (vitamin D₃ analogs with alkynyl, alkenyl and alkanyl side chains); U.S. Pat. No. 5,120,722 (trihydroxycalciferol); U.S. Pat. No. 5,547,947 (fluorocholecalciferol compounds); U.S. Pat. No. 5,446,035 (methyl substituted vitamin D); U.S. Pat. No. 5,411,949 (23-oxa-derivatives); U.S. Pat. No. 5,237,110 (19-nor-vitamin D compounds); U.S. Pat. No. 4,857,518 (hydroxylated 24-homo-vitamin D derivatives). Additional examples of active vitamin D compounds are listed in the following patents, each of which is incorporated by reference herein in its entirety: U.S. Pat. Nos. 6,503,893, 6,482,812, 6,441,207, 6,410,523, 6,399,797, 6,392,071, 6,376,480, 6,372,926, 6,372,731, 6,359,152, 6,329,357, 6,326,503, 6,310,226, 6,288,249, 6,281,249, 6,277,837, 6,218,430, 6,207,656, 6,197,982, 6,127,559, 6,103,709, 6,080,878, 6,075,015, 6,072,062, 6,043,385, 6,017,908, 6,017,907, 6,013,814, 5,994,332, 5,976,784, 5,972,917, 5,945,410, 5,939,406, 5,936,105, 5,932,565, 5,929,056, 5,919,986, 5,905,074, 5,883,271, 5,880,113, 5,877,168, 5,872,140, 5,847,173, 5,843,927, 5,840,938, 5,830,885, 5,824,811, 5,811,562, 5,786,347, 5,767,111, 5,756,733, 5,716,945, 5,710,142, 5,700,791, 5,665,716, 5,663,157, 5,637,742, 5,612,325, 5,589,471, 5,585,368, 5,583,125, 5,565,589, 5,565,442, 5,554,599, 5,545,633, 5,532,228, 5,508,392, 5,508,274, 5,478,955, 5,457,217, 5,447,924, 5,446,034, 5,414,098, 5,403,940; 5,397,775; 5,395,830; 5,393,749; 5,384,313; 5,374,629; 5,373,004; 5,371,249; 5,321,018; 5,281,731; 5,260,290; 5,254,538; 5,250,523; 5,247,104; 5,246,925; 5,232,836; 5,194,431; 5,185,150 5,086,191; 5,036,061; 5,030,772; 4,973,584; 5,354,744; 4,940,700; 4,927,815; 4,866,048; 4,851,400; 4,847,012; 4,804,502; 4,769,181; 4,755,329; 4,719,205; 4,719,204; 4,619,920; 4,594,192; 4,588,716; 4,588,528; 4,564,474; 4,552,698; 4,689,180; 4,505,906; 4,502,991; 4,481,198; 4,448,726; 4,448,721; 4,428,946; 4,411,833; 4,367,177; 4,360,472; 4,360,471; 4,358,406; 4,336,193; 4,307,231; 4,307,025; 4,305,880; 4,279,826; and, 4,248,791. A more comprehensive list of active vitamin D compounds can be found in published PCT Application No. WO 99/49870, which is incorporated by reference herein in its entirety.

Other active vitamin D compounds in clinical use include but are not limited to investigational drugs from Leo Pharmaceutical such as EB 1089 (24a,26a,27a-trihomo-22,24-diene-1αa,25-(OH)₂-D₃), KH 1060 (20-epi-22-oxa-24a,26a,27a-trihomo-1α,25-(OH)₂-D₃), MC 1288 and MC 903 (calcitriol); Roche Pharmaceutical drugs such as 1,25-(OH)2-16-ene-D3, 1,25-(OH)₂-16-ene-23-yne-D₃ and 25-(OH)₂-16-ene-23-yne-D₃; Chugai Pharmaceuticals such as 22-oxacalcitriol (22-oxa-1α,25-(OH)-2-D₃); University of Illinois such as 1α-(OH)D₅; and the Institute of Medical Chemistry-Schering AG such as ZK 161422 (20-methyl-1,25-(OH)₂-D₃) and ZK 157202 (20-methyl-23-ene-1,25-(OH)₂-D₃). Vitamin D analogs also include topical preparations of vitamin D compounds, such as calcipotriene (DOVONEX®) and Tacalcitol (CURATODERM®). Examples of particular commercially available active vitamin D compound formulations are ROCALTROL®, which is available from Roche; and CALCIJEX®, which is available from Abbott.

Additional examples of active vitamin D compounds and their derivatives, analogs, homologs, precursors and metabolites include but are not limited to 1α,25-(OH)₂-26,27-d₆-D₃; 1α,25-(OH)₂-22-ene-D₃; 1α-(OH)₂-D₃; 1α,25-(OH)₂-D₂; 1α,25-(OH)₂-D₄; 1α,24,25-(OH)₃-D₃; 1α,24,25-(OH)₃-D₂; 1α,24,25-(OH)₃-D₄; 1α-(OH)-25-FD₃; 1α-(OH)-25-FD₄; 1α(OH)-25-FD₂; 1α,24-(OH)-2-D₄; 1α,24-(OH)₂-D₃; 1α,24-(OH)₂-D₂; 1α,24-(OH)₂-25-FD₄; 1α, 24-(OH)₂-25-FD₃; 1α,24-(OH)₂-25-FD₂; 1α,25-(OH)₂-26,27-F₆-22-ene-D₃; 1α,25-(OH)₂-26,27-F₆-D₃; 1α,25S—(OH)₂-26-F₃-D₃; 1α,25-(OH)₂-24-F₂-D₃; 1α,25S,26-(OH)₂-22-ene-D₃; 1α,25R,26-(OH)₂-22-ene-D₃; 1α,25-(OH)-2-D₂; 1α,25-(OH)₂-24-epi-D₃; 1α,25-(OH)₂-23-yne-D₃; 1α,25-(OH)₂-24R—F-D₃; 1α,25S,26-(OH)₂-D₃; 1α,24R—(OH)₂-25F-D₃; 1α,25-(OH)₂-26,27-F₆-23-yne-D₃; 1α,25R—(OH)₂-26-F₃-D₃; 1α,25,28-(OH)-3-D₂; 1α,25-(OH)₂-16-ene-23-yne-D₃; 1α,24R,25-(OH)₃-D₃; 1α,25-(OH)₂-26,27-F₆-23-ene-D₃; 1α,25R—(OH)₂-22-ene-26-F₃-D₃; 1α,25S—(OH)₂-22-ene-26-F₃-D₃; 1α,25R—(OH)₂-D₃-26,26,26-d₃; 1α,25S—(OH)₂-D₃-26,26,26-d₃; and 1α,25R—(OH)₂-22-ene-D₃-26,26,26-d₃.

Further, while any vitamin D compound may be used according to the methods of the invention, preferred vitamin D compounds have pharmacokinetic properties that make them more suitable for the below-described methods than other vitamin D compounds. In general, preferred vitamin D compounds achieve peak plasma concentrations rapidly, e.g., within about four hours, and are eliminated quickly, e.g., with an elimination half-life of about 12 hours or fewer. The elimination half-life describes the time for the plasma concentration of the agent to be reduced by 50%, while eliminated in this context is meant to refer to the plasma concentrations below about 0.5 nM. While endogenous vitamin D plasma concentrations vary from subject to subject, they are typically about 0.16 nM. Calcitriol is an example of such a preferred vitamin D compound with desirable pharmacokinetic properties as described above. While not intending to be bound to any particular theory or mechanism of action, it is believed that vitamin D compounds with these pharmacokinetic properties can initiate the therapeutic biological response during the brief period of elevated concentration, then quickly fall below the threshold concentration that facilitates calcium release, thereby minimizing hypercalcemia.

In preferred embodiments of the invention, the active vitamin D compound is calcitriol.

Non-Hypercalcemic Vitamin D Compounds

The vitamin D compounds used in the present invention also comprise non-hypercalcemic vitamin D compounds. However, in certain embodiments of the invention, the vitamin D compound is not a non-hypercalcemic vitamin D compound. Non-hypercalcemic vitamin D compounds have less of a tendency to produce the onset of hypercalcemia than a comparable dosage of calcitriol as assessed by assays well-known to one of skill in the art.

Examples of such non-hypercalcemic vitamin D compounds include analogs of calcitriol such as Ro23-7553 and Ro24-5531 (1α,25-dihydroxy-16-ene-23-yne-26,27-hexafluorocholecalciferol) available from Hoffmann-LaRoche. Other examples of non-hypercalcemic vitamin D compounds can be found in U.S. Pat. No. 4,717,721, which is incorporated by reference herein in its entirety.

The foregoing description of vitamin D compounds is not exhaustive and is merely exemplary of all compounds capable of binding to VDRs. One of skill in the art will appreciate that this invention encompasses all vitamin D compounds, i.e. all compounds capable of binding to VDRs, and the derivatives, analogs, homologs, precursors, metabolites, and pharmaceutically acceptable salts, solvates, hydrates, stereoisomers, clathrates and prodrugs thereof.

### Other Therapeutic Agents

In certain aspects, the present invention provides compositions for treatment of MDS, or amelioration of one or more symptoms thereof, by administration of a vitamin D compound in combination with one or more additional active agent(s). The additional active agent can be any active agent having a therapeutic effect to treat MDS, or ameliorate a symptom thereof, that is known to one of skill in the art without limitation. Active agents include, but are not limited to, small molecules, synthetic drugs, peptides, polypeptides, proteins, nucleic acids (e.g., DNA and RNA nucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. Any agent which is known to be useful, or which has been used or is currently being used for the treatment of MDS, or amelioration of one or more symptoms associated with MDS, can be used in combination with a vitamin D compound in accordance with the invention described herein.

In certain embodiments, the compositions of the invention encompass administration of a vitamin D compound of the invention in conjunction with one or more additional active agents that have combinatorial, synergistic, additive, or other therapeutic effects. In one embodiment, a vitamin D compound of the invention can be administered with a growth factor such as a cytokine or hematopoietic growth factor. In another embodiment, a vitamin D compound of the invention can be administered with an immunomodulator. In yet another embodiment, a vitamin D compound of the invention can be administered with a cytotoxic agent. In yet another embodiment, a vitamin D compound of the invention may be administered with an agent that affects RNA transcription. In still another embodiment, a vitamin D compound of the invention can be administered with a derivative of vitamin A, E, or K. In yet another embodiment, a vitamin D compound of the invention can be administered with an agent that specifically binds biological targets related to MDS. In still another embodiment, a vitamin D compound of the invention can be administered with a signal transduction inhibitor. In yet another embodiment, a vitamin D compound of the invention can be administered with an aminothiol. In still another embodiment, a vitamin D compound of the invention can be administered with an arsenic-containing compound. Further embodiments of the invention encompass administration of vitamin D compound of the invention in conjunction with more than one of the active agents described herein.

Growth Factors or Cytokines

In one embodiment of the invention, a vitamin D compound of the invention can be administered with a growth factor. Any growth factor known by one of skill in the art to be effective to treat MDS, or ameliorate a symptom thereof, may be administered with a vitamin D compound to a subject in need of such administration. In a further embodiment of the invention, one or more growth factor(s) known by one of skill in the art to be effective to treat MDS, or ameliorate a symptom thereof, may be administered with a vitamin D compound and one or more additional active agent(s) as described herein.

Examples of growth factors that can be used in the various embodiments of the invention, including pharmaceutical compositions, dosage forms, and kits of the invention, include, without limitation, cytokines or hematopoietic growth factors such as, e.g., EPO, TPO, GM-CSF, G-CSF, IFN-alpha, IL-1, IL-2, IL-3, IL-6, IL-8, IL-11, and IL-12. Furthermore, recombinant, modified, mimetic, fragmentary, or analogous forms of the above described cytokines or hematopoietic growth factors may also be used in the various embodiments of the invention. See, e.g., U.S. Pat. Nos. 6,358,505, 6,346,531, 6,340,742, 6,262,253, 6,261,550, 6,166,183, 6,100,070, 5,986,047, 5,981,551, 5,916,773, 5,902,584, 5,835,382, 5,824,778, 5,773,581, 5,773,569, and 5,756,349, all of which describe recombinant, modified, mimetic, fragmentary, or analogous forms of EPO and G-CSF and each of which is incorporated herein by reference in its entirety. Preferred cytokines or hematopoietic growth factors include r-HuEPO and r-metHuG-CSF.

An example of a commercial form of r-HuEPO is EPOGEN®, which is produced by recombinant DNA technology and has the same biological effects and the same amino acid sequence as endogenous erythropoietin. A 1 ml dosage form of EPOGEN® can contain 2000, 3000, 4000, or 10,000 Units of epoetin alfa, 2.5 mg albumin (human), 1.2 mg sodium phosphate monobasic monohydrate, 1.8 mg sodium phosphate dibasic anhydrate, 0.7 mg sodium citrate, 5.8 mg sodium chloride, and 6.8 mg of citric acid, in water for injection, USP (pH 6.9±0.3). Multidose forms of EPOGEN® are available, and all dosage forms are in vials for parenteral administration. See Physicians' Desk Reference 582 (56th ed., 2002).

An example of a commercial form of r-metHuG-CSF, also known as filgrastim, is NEUPOGEN®, which is produced by recombinant DNA technology in E. coli and differs from G-CSF isolated from human cells in that it is not glycosylated. A 1 ml dosage form of NEUPOGEN® can contain 300 μg of filgrastim, 0.59 mg Acetate, 50.0 mg Sorbitol, 0.004% TWEEN® 80, 0.035 mg Sodium and 1.0 mL Water for Injection, USP. Larger dosage forms of NEUPOGEN® are available, and all dosage forms are in vials for parenteral administration. See Id. at 588.

Immunomodulators

In another embodiment of the invention, a vitamin D compound of the invention can be administered with an immunomodulator. The immunomodulator can be any immunomodulator known by one of skill in the art to be effective to treat MDS, or ameliorate a symptom thereof. In a further embodiment of the invention, one or more immunomodulator(s) known by one of skill in the art to be effective to treat MDS, or ameliorate a symptom thereof, may be administered to a subject with a vitamin D compound and one or more additional active agent(s) described herein.

Examples of immunomodulators that can be used in the various embodiments of the invention, including pharmaceutical compositions, dosage forms, and kits of the invention, include, without limitation, anti-thymocyte globulin (ATG), anti-lymphocyte globulin (ALG), thalidomide, prednisone, cyclosporin A (CyA), dexamethasone, and pentoxifylline.

Cytotoxic Agents

In yet another embodiment of the invention, a vitamin D compound of the invention can be administered with a cytotoxic agent. The cytotoxic agent can be any cytotoxic agent known by one of skill in the art to be effective to treat MDS, or ameliorate a symptom thereof. In a further embodiment of the invention, one or more cytotoxic agent(s) known by one of skill in the art to be effective to treat MDS, or ameliorate a symptom thereof, may be administered to a subject with a vitamin D compound and one or more additional active agent(s) as described herein.

Any cytotoxic agent can be employed according to the methods of the invention; many cytotoxic agents suitable for chemotherapy of MDS or cancer in general are known in the art. For example, the cytotoxic agent can be an anti-metabolite (e.g., 5-flourouricil (5-FU), methotrexate (MTX), fludarabine, etc.), an anti-microtubule agent (e.g., vincristine; vinblastine; taxanes such as paclitaxel and docetaxel; etc.), an alkylating agent (e.g., cyclophosphamide, melphalan, bischloroethylnitrosurea, etc.), platinum agents (e.g., cisplatin, carboplatin, oxaliplatin, JM-216, CI-973, etc.), anthracyclines (e.g., doxorubicin, daunorubicin, etc.), antibiotic agents (e.g., mitomycin-C, actinomycin D, etc.), topoisomerase inhibitors (e.g., etoposide, camptothecins, etc.), or other cytotoxic agents.

Particular examples of cytotoxic agents that can be used in the various embodiments of the invention, including pharmaceutical compositions, dosage forms, and kits of the invention, include, without limitation, cytarabine, melphalan, topotecan, fludarabine, etoposide, idarubicin, daunorubicin, mitoxantrone, cisplatin, paclitaxel, and cyclophosphamide.

Other chemotherapeutic agents that may be used include abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate

Other Therapeutic Agents for MDS

In yet another embodiment of the invention, a vitamin D compound of the invention can be administered with an agent that affects RNA transcription. The agent that affects RNA transcription can be any agent that affects RNA transcription known by one of skill in the art to be effective to treat MDS, or ameliorate a symptom thereof. In a further embodiment of the invention, one or more agent(s) that affect RNA transcription known by one of skill in the art to be effective to treat MDS, or ameliorate a symptom thereof, may be administered to a subject with a vitamin D compound and one or more additional active agent(s) as described herein. Non-limiting examples of an agent that affects RNA transcription that can be used in the various embodiments of the invention, including pharmaceutical compositions, dosage forms, and kits of the invention, include decitabine, 5-azacytidine, depsipeptides, and phenylbutyrate.

In yet another embodiment of the invention, a vitamin D compound of the invention can be administered with a derivative of vitamin A, E, or K. The derivative of vitamin A, E, or K can be any derivative of vitamin A, E, or K known by one of skill in the art to be effective to treat MDS, or ameliorate a symptom thereof. In a further embodiment of the invention, one or more derivative(s) of vitamin A, E, or K known by one of skill in the art to be effective to treat MDS, or ameliorate a symptom thereof, may be administered to a subject with a vitamin D compound and one or more additional active agent(s) as described herein, Non-limiting examples of a derivative of vitamin A, E, or K that can be used in the various embodiments of the invention, including pharmaceutical compositions, dosage forms, and kits of the invention, include all trans retinoic acid, 13-cis-retinoic acid, tocopherol, and menatetrenone.

In still another embodiment of the invention, a vitamin D compound of the invention can be administered with an agent that specifically binds biological targets related to MDS. The agent that specifically binds biological targets related to MDS can be any agent that specifically binds biological targets related to MDS known by one of skill in the art to be effective to treat MDS, or ameliorate a symptom thereof. In a further embodiment of the invention, one or more agent(s) that specifically bind biological targets related to MDS known by one of skill in the art to be effective to treat MDS, or ameliorate a symptom thereof, may be administered to a subject with a vitamin D compound and one or more additional active agent(s) as described herein. Non-limiting examples of an agent that specifically binds biological targets related to MDS that can be used in the various embodiments of the invention, including pharmaceutical compositions, dosage forms, and kits of the invention, include anti-VEGF, gemtuzumab ozogamicin, and TNFR:Fc.

In still another embodiment of the invention, a vitamin D compound of the invention can be administered with a signal transduction inhibitor. The signal transduction inhibitor can be any signal transduction inhibitor known by one of skill in the art to be effective to treat MDS, or ameliorate a symptom thereof. In a further embodiment of the invention, one or more signal transduction inhibitor(s) known by one of skill in the art to be effective to treat MDS, or ameliorate a symptom thereof, may be administered to a subject with a vitamin D compound and one or more additional active agent(s) as described herein. Non-limiting examples of such signal transduction inhibitors that can be used in the various embodiments of the invention, including pharmaceutical compositions, dosage forms, and kits of the invention, include farnesyl transferase inhibitors such as, e.g. ZARNESTRA™ and SARASAR™ and tyrosine kinase inhibitors such as, e.g., SU5416, SU6668, and PTK787/ZK222584.

In still another embodiment of the invention, a vitamin D compound of the invention can be administered with an aminothiol. The aminothiol can be any aminothiol known by one of skill in the art to be effective to treat MDS, or ameliorate a symptom thereof. In a further embodiment of the invention, one or more aminothiol(s) known by one of skill in the art to be effective to treat MDS, or ameliorate a symptom thereof, may be administered to a subject with a vitamin D compound and one or more additional active agent(s) as described herein. A non-limiting example of an aminothiol that can be used in the various embodiments of the invention, including pharmaceutical compositions, dosage forms, and kits of the invention, is amifostine.

In still another embodiment of the invention, a vitamin D compound of the invention can be administered with an arsenic-containing compound. The arsenic-containing compound can be any arsenic-containing compound known by one of skill in the art to be effective to treat MDS, or ameliorate a symptom thereof. In a further embodiment of the invention, one or more arsenic-containing compound(s) known by one of skill in the art to be effective to treat MDS, or ameliorate a symptom thereof, may be administered to a subject with a vitamin D compound and one or more additional active agent(s) as described herein. A non-limiting example of an arsenic-containing compound that can be used in the various embodiments of the invention, including pharmaceutical compositions, dosage forms, and kits of the invention, is arsenic trioxide.

### Methods of Treating Myelodysplastic Syndromes

The invention provides methods of treating MDS, or ameliorating a symptom thereof, in a subject in need of such treatment or amelioration. It further encompasses methods of treating subjects who have been previously treated for MDS, as well as those who have not previously been treated for MDS. Because subjects with MDS have heterogeneous clinical manifestations and varying clinical outcome, it has become apparent that staging the subjects according to their prognosis and approaching therapy depending on the severity and stage is necessary. Indeed, the methods of this invention can be used in various stages of treatments for subjects with one or more types of MDS including but not limited to refractory anemia (RA), RA with ringed sideroblasts (RARS), RA with excess blasts (RAEB), RAEB in transformation (RAEB-T), or chronic myelomonocytic leukemia (CMML). In addition, the methods of the invention can be used to in various stages of treatments for subjects with one or more types of MDS including but not limited to low risk, intermediate-1 risk, intermediate-2 risk, or high risk MDS.

The present invention provides methods for administering therapeutically effective doses of vitamin D compounds while minimizing the risk of hypercalcemia for the treatment of myelodysplastic syndromes, or amelioration of a symptom thereof. In certain embodiments, the methods comprise administering a therapeutically effective dose of vitamin D compounds in the treatment of MDS, or amelioration of a symptom thereof. In further embodiments, the methods incorporate administering the vitamin D compounds intermittently in high doses. Intermittent administration of the vitamin D compounds allows the high doses to be administered to a subject while minimizing or eliminating hypercalcemia. In still further embodiments, the methods incorporate the use of oral vitamin D compound formulations. In yet other embodiments, the methods incorporate the use of stable, oral vitamin D compound formulations with improved bioavailability and rapid onset of peak blood levels of vitamin D compounds. In still other embodiments, the methods incorporate the use of oral vitamin D compound formulations in the form of an emulsion pre-concentrate. In yet other embodiments, the methods incorporate the use of intravenous (i.v.) vitamin D compound formulations. In certain embodiments, the vitamin D compounds are administered as a monotherapy. In other embodiments, the vitamin D compounds and formulations are administered in combination with one or more additional active agents. In yet other embodiments, the vitamin D compounds and formulations are administered in combination with one or more hematopoietic growth factors or cytokines.

In certain embodiments, the methods of the invention encompass administering a therapeutically effective dose of vitamin D compounds while minimizing the risk of hypercalcemia for the treatment of myelodysplastic syndromes, or amelioration of a symptom thereof. In certain embodiments, the methods of the invention encompass administering a therapeutically effective dose of vitamin D compounds for the treatment of anemia associated with MDS. In other embodiments, the methods of the invention encompass administering a therapeutically effective dose of vitamin D compounds to increase plasma hemoglobin concentrations of a subject with MDS. In yet other embodiments, the methods of the invention encompass administering a therapeutically effective dose of vitamin D compounds to reduce transfusion requirements of a subject with MDS. In still other embodiments, the methods of the invention encompass administering a therapeutically effective dose of vitamin D compounds for the treatment of thrombocytopenia associated with MDS. In yet other embodiments, the methods of the invention encompass administering a therapeutically effective dose of vitamin D compounds to reduce the fatigue of a subject with MDS. In still other embodiments, the methods of the invention encompass administering a therapeutically effective dose of vitamin D compounds to decrease the frequency and severity of bruising of a subject with MDS. In yet other embodiments, the methods of the invention encompass administering a therapeutically effective dose of vitamin D compounds to reduce the frequency and severity of bleeding episodes of a subject with MDS. In yet other embodiments, the methods of the invention encompass administering a therapeutically effective dose of vitamin D compounds to reduce the frequency and severity of fevers suffered by a subject with MDS. In still other embodiments, the methods of the invention encompass administering a therapeutically effective dose of vitamin D compounds for the treatment of neutropenia associated with MDS. In still other embodiments, the methods of the invention encompass administering a therapeutically effective dose of vitamin D compounds to reduce the frequency and severity of infections in a subject with MDS. In yet other embodiments, the methods of the invention encompass administering a therapeutically effective dose of vitamin D compounds to delay the progression of MDS to leukemia in a subject with MDS. In still other embodiments, the methods of the invention encompass administering a therapeutically effective dose of vitamin D compounds to extend the survival of a subject with MDS.

Without intending to be bound by any particular theory or mechanism of action, it is believed that vitamin D compounds and other therapeutic agents effective to treat MDS can act in complementary or synergistic ways in the treatment of MDS, or amelioration of a symptom thereof. Therefore, one embodiment of the invention encompasses a method of treating MDS, or ameliorating a symptom thereof, which comprises administering to a patient in need of such treatment and/or amelioration a therapeutically effective dose of a vitamin D compound, or a pharmaceutically acceptable salt, solvate, hydrate, stereoisomer, clathrate, or prodrug thereof and a therapeutically effective dose of one or more additional active agent(s) as described herein.

One of skill in the art will recognize that any of the methods of treatment described herein may further be used for the prevention of the onset or recurrence of MDS in a subject in whom such prevention is desired.

Administration and Dosage of Vitamin D Compounds in Treatment of Myelodysplastic Syndrome

High systemic levels of vitamin D compounds can be achieved without the onset of hypercalcemia by intermittently administering the vitamin D compounds according to the methods of the invention. High doses of vitamin D compounds include doses greater than about 3 μg as discussed in the sections below. Therefore, in certain embodiments of the invention, the methods for the treatment of MDS, or amelioration of a symptom thereof, encompass intermittently administering high doses of vitamin D compounds. High doses of vitamin D compounds can be administered before, concurrently with, after, or in cycles with other therapies, including but not limited to pharmacotherapy. The administration of vitamin D compounds can also occur in cycling regimens such that administration of the vitamin D compound can occur before, concurrently with, after, or in any cycling regimen with other treatments within a cycling series of such treatments. The frequency of the intermittent administration can be limited by a number of factors including but not limited to the pharmacokinetic parameters of the compound or formulation and the pharmacodynamic effects of the vitamin D compound on the subject. For example, subjects with MDS having impaired renal function may require less frequent administration of the vitamin D compounds because of those subjects' decreased ability to excrete calcium,

The following is exemplary only and merely serves to illustrate that the term “intermittent” can encompass any administration regimen designed by a person of ordinary skill in the art.

In one example, the vitamin D compound can be administered not more than once every three days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In another example, the vitamin D compound can be administered not more than once every four days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In still another example, the vitamin D compound can be administered not more than once every five days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In yet another example, the vitamin D compound can be administered not more than once every six days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In still another example, the vitamin D compound can be administered not more than once every seven days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In yet another example, the vitamin D compound can be administered not more than once every eight days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In still another example, the vitamin D compound can be administered not more than once every nine days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In yet another example, the vitamin D compound can be administered not more than once every ten days. The administration can continue for one, two, three, or four weeks or one, two, three, four, five, or six months, or one year, or longer. In certain embodiments, the vitamin D compound can be administered until the anemia associated with MDS is ameliorated. Optionally, after a period of rest, the vitamin D compound can be administered under the same or different schedule. The period of rest can be one, two, three, or four weeks, or longer, according to the pharmacodynamic effects of the vitamin D compound on the subject.

In yet another example, the vitamin D compound can be administered once per week for three months. Optionally, after a period of rest, the vitamin D compound can be administered under the same or different schedule. The period of rest can be one, two, three, or four weeks, or longer, according to the pharmacodynamic effects of the vitamin D compound on the subject.

In still another example, the vitamin D compound can be administered once every three weeks for a year. Optionally, after a period of rest, the vitamin D compound can be administered under the same or different schedule. The period of rest can be one, two, three, or four weeks, or longer, according to the pharmacodynamic effects of the vitamin D compound on the subject.

In a preferred example, the vitamin D compound can be administered once per week for 3 weeks out of each 4 week cycle. After a one week period of rest, the vitamin D compound can be administered under the same or different schedule.

Further examples of dosing schedules that can be used in the methods of the present invention are provided in U.S. Pat. No. 6,521,608, which is incorporated by reference in its entirety.

The above-described administration schedules are provided for illustrative purposes only and should not be considered limiting. A person of ordinary skill in the art will readily understand that all vitamin D compounds are within the scope of the invention; that calcitriol and its analogs, homologs, derivatives, precursors and metabolites are preferred; and that the exact dosing and schedule of administration of the vitamin D compounds can vary due to many factors.

The amount of a therapeutically effective dose of a pharmaceutical agent in the acute or chronic management of a disease or disorder may vary depending on factors including, but not limited to, the disease or disorder treated, the specific pharmaceutical agents and the route of administration. According to the methods of the invention, a therapeutically effective dose of a vitamin D compound is any dose of the vitamin D compound effective to treat MDS or ameliorate a symptom thereof. A high dose of a vitamin D compound can be a dose from about 3 μg to about 300 μg or any dose within this range as discussed below. The dose, dose frequency, duration, or any combination, may also vary according to age, body weight, response, and the past medical history of the subject as well as the route of administration, pharmacokinetic and pharmacodynamic effects of the pharmaceutical agent. These factors are routinely considered by one of skill in the art.

The rates of absorption and clearance of vitamin D compounds are affected by a variety of factors that are well-known to persons of ordinary skill in the art. As discussed above, the pharmacokinetic properties of vitamin D compounds limit the peak concentration of vitamin D compounds that can be obtained in the blood without inducing the onset of hypercalcemia and preferably without inducing the onset of clinical hypercalcemia. The rate and extent of absorption, distribution, binding or localization in tissues, biotransformation and excretion of the vitamin D compound can all affect the frequency at which the pharmaceutical agent can be administered. In certain embodiments, vitamin D compounds are administered intermittently in high doses as a method of treating MDS, or ameliorating a symptom thereof, according to the dosing schedule described above.

In certain embodiments of the invention, the methods comprise administering a vitamin D compound, or a pharmaceutically acceptable salt, solvate, hydrate, stereoisomer, clathrate, or prodrug thereof in a dose of from about 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, or 300 μg, or any range of doses therein. In certain embodiments, the methods of the invention comprise administering a dose of a vitamin D compound, or a pharmaceutically acceptable salt, solvate, hydrate, stereoisomer, clathrate, or prodrug thereof in a dose of from about 0.12 μg/kg to about 3 μg/kg. In other embodiments, the methods of the invention comprise administering a dose of a vitamin D compound, or a pharmaceutically acceptable salt, solvate, hydrate, stereoisomer, clathrate, or prodrug thereof in a dose of from about 3 μg to about 300 μg. In yet other embodiments, the methods of the invention comprise administering a dose of a vitamin D compound, or a pharmaceutically acceptable salt, solvate, hydrate, stereoisomer, clathrate, or prodrug thereof in a dose of from about 5 μg to about 200 μg. In still other embodiments, the methods of the invention comprise administering a dose of a vitamin D compound, or a pharmaceutically acceptable salt, solvate, hydrate, stereoisomer, clathrate, or prodrug thereof in a dose of from about 5 μg to about 105 μg. In still other embodiments, the methods of the invention comprise administering a dose of a vitamin D compound, or a pharmaceutically acceptable salt, solvate, hydrate, stereoisomer, clathrate, or prodrug thereof in a dose of from about 15 μg to about 105 μg. In yet other embodiments, the methods of the invention comprise administering a dose of a vitamin D compound, or a pharmaceutically acceptable salt, solvate, hydrate, stereoisomer, clathrate, or prodrug thereof in a dose of from about 15 μg to about 90 μg. In still other embodiments, the methods of the invention comprise administering a dose of a vitamin D compound, or a pharmaceutically acceptable salt, solvate, hydrate, stereoisomer, clathrate, or prodrug thereof in a dose of from about 20 μg to about 80 μg. In yet other embodiments, the methods of the invention comprise administering a dose of a vitamin D compound, or a pharmaceutically acceptable salt, solvate, hydrate, stereoisomer, clathrate, or prodrug thereof in a dose of from about 30 μg to about 60 μg. In still other embodiments, the methods of the invention comprise administering a dose of a vitamin D compound, or a pharmaceutically acceptable salt, solvate, hydrate, stereoisomer, clathrate, or prodrug thereof in a dose of from about 30 μg to about 75 μg. In a preferred embodiment, the methods of the invention comprise administering a dose of a vitamin D compound, or a pharmaceutically acceptable salt, solvate, hydrate, stereoisomer, clathrate, or prodrug thereof in a dose of about 45 μg. One of skill in the art will recognize that these standard doses are for an average sized adult of approximately 70 kg and can be adjusted for the factors routinely considered as stated above. While not intending to be bound by any particular theory or mechanism of action, it is believed that doses of the vitamin D compounds of up to 105 μg may be administered without substantially increasing the half-life and associated toxicity of the vitamin D compounds. Therefore, in a preferred embodiment, the dose of the vitamin D compound is 105 μg or less.

In certain embodiments, the methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 0.1 nM, 0.2 nM, 0.3 nM, 0.4 nM, 0.5 nM, 0.6 nM, 0.7 nM, 0.8 nM. 0.9 nM, 1 nM, 2 nM, 3 nM, 4 nM, 5 nM, 6 nM, 7 nM, 8 nM, 9 nM, 10 nM, 12 nM, 15 nM, 17 nM, or 20 nM, or any range of concentrations therein. In other embodiments, the methods of the invention comprise administering a dose of a vitamin D compound or a pharmaceutically acceptable salt, solvate, hydrate, stereoisomer, clathrate, or prodrug thereof in a dose that achieves peak plasma concentrations of the vitamin D compound exceeding about 0.5 nM. In other embodiments, methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 0.5 nM to about 20 nM. In other embodiments, the methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 1 nM to about 10 nM. In still other embodiments, methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 1 nM to about 7 nM. In yet other embodiments, methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 3 nM to about 7 nM. In yet other embodiments, methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 5 nM to about 7 nM. In a preferred embodiment, methods of the invention comprise administering a dose of a vitamin D compound, or a pharmaceutically acceptable salt, solvate, hydrate, stereoisomer, clathrate, or prodrug thereof in a dose that achieves peak plasma concentrations of the vitamin D compound from about 3 nM to about 5 nM.

In certain embodiments, the methods of the invention further comprise administering a dose of a vitamin D compound, or a pharmaceutically acceptable salt, solvate, hydrate, stereoisomer, clathrate, or prodrug thereof that achieves peak plasma concentrations rapidly, e.g., within four hours. In further embodiments, the methods of the invention comprise administering a dose of a vitamin D compound, or a pharmaceutically acceptable salt, solvate, hydrate, stereoisomer, clathrate, or prodrug thereof that is eliminated quickly, e.g., with an elimination half-life of less than 12 hours.

In other aspects, the methods of the invention encompass intermittently administering high doses of vitamin D compounds to a subject with MDS and monitoring the subject for symptoms associated with hypercalcemia. In certain embodiments, the methods of the invention encompass intermittently administering high doses of vitamin D compounds to a subject with MDS and monitoring the renal function of the subject. In other embodiments, the methods of the invention encompass intermittently administering high doses of vitamin D compounds to a subject with MDS and monitoring the subject for calcification of soft tissues, such as, for example, cardiac tissue. In still other embodiments, the methods of the invention encompass intermittently administering high doses of vitamin D compounds to a subject with MDS and monitoring the subject for increased bone density. In yet other embodiments, the methods of the invention encompass intermittently administering high doses of vitamin D compounds to a subject with MDS and monitoring the subject for hypercalcemic nephropathy. In still other embodiments, the methods of the invention encompass intermittently administering high doses of vitamin D compounds to a subject with MDS and monitoring the blood calcium concentration of the subject to ensure that the blood calcium concentration is less than about 10.5 mg/dL.

In certain embodiments, high blood levels of vitamin D compounds can be safely obtained in conjunction with reducing the transport of calcium into the blood. In one embodiment, higher 1,25-dihydroxyvitamin D concentrations are safely obtainable without the onset of hypercalcemia when administered in conjunction with a reduced calcium diet. In one example, the calcium can be trapped by an adsorbent, absorbent, ligand, chelate, or other binding moiety that cannot be transported into the blood through the small intestine. In another example, the rate of osteoclast activation can be inhibited by administering, for example, a bisphosphonate such as, e.g., pamidronate, or ZOMETA (Novartis Pharmaceuticals Corp., East Hanover, N.J.) in conjunction with the vitamin D compound.

In certain embodiments, high blood levels of vitamin D compounds are safely obtained in conjunction with maximizing the rate of clearance of calcium. In one example, calcium excretion can be increased by ensuring adequate hydration and salt intake. In another example, diuretic therapy can be used to increase calcium excretion.

Administering Vitamin D Compounds in Combination with Other Therapeutic Agents

The methods of the present invention also provide combination therapies comprising administering one or more vitamin D compounds in combination with one or more additional active agents that are not vitamin D compounds. The additional active agents can be any agents that have a therapeutic effect to treat MDS, or ameliorate a symptom thereof, that are known to one of skill in the art without limitation. Proposed mechanisms for these active agents can be found in the art (see, e.g., Hardman et al., eds., 1996, Goodman & Gilman's The Pharmacological Basis of Therapeutics 10^{th} Ed, McGraw-Hill, New York at pages 643-754, 1381-1484, 1649-1678, and Physician's Desk Reference (PDR) 55^{th} Ed., 2001, Medical Economics Co., Inc., Montvale, N.J. In certain embodiments, the combination therapies of the present invention comprise administering one or more additional active agents which improve the therapeutic or ameliorative effects of the vitamin D compounds by producing an additive or synergistic effect.

In accordance with the present invention, the combination therapies are advantageously utilized for the treatment of MDS, or amelioration of a symptom thereof. The one or more vitamin D compounds may be administered prior to (e.g., 0.5 hours, 1 hour, 2 hours, 4 hours, 6 hours, 12 hours, 24 hours, 36 hours, 48 hours, 5 days, 1 week, 2 weeks, 1 month or more before), after (e.g., 0.5 hours, 1 hour, 2 hours, 4 hours, 6 hours, 12 hours, 24 hours, 36 hours, 48 hours, 5 days, 1 week, 2 weeks, 1 month or more after), concurrently with, or in any cycling regimen involving the administration of one or more additional active agents. In embodiments where the additional active agents are administered less often than the vitamin D compounds, the one or more vitamin D compounds are preferably administered about one day before the one or more additional active agents.

In certain embodiments, the additional active agents that are not vitamin D compounds can be one or more growth factors. The growth factors can be administered before, concurrently with, after, or in cycles with a vitamin D compound in the methods of the present invention to benefit from the ability of the vitamin D compound to sensitize cells to the actions of the growth factors. Thus, less growth factor may be used in the treatment of MDS, or amelioration of a symptom thereof. In other embodiments, cycling therapy is used to inhibit the development of resistance or reduce the resistance to one or more of the therapies, avoid or reduce the side effects of the therapies, and/or improve the effectiveness of the treatments.

In certain embodiments, the one or more growth factors can be cytokines. The cytokines can be administered before, concurrently with, after, or in cycles with a vitamin D compound according to the methods of the present invention. In other embodiments, the one or more growth factors can be hematopoietic growth factors. The hematopoietic growth factors can be administered before, concurrently with, after, or in cycles with a vitamin D compound according to the methods of the present invention.

In certain embodiments, the hematopoietic growth factor administered before, concurrently with, after, or in cycles with a vitamin D compound can be EPO, e.g., r-HuEPO, or a pharmacologically active mutant or derivative thereof. In other embodiments, the hematopoietic growth factor that can be administered before, concurrently with, after, or in cycles with a vitamin D compound can be G-CSF, e.g., r-metHuG-CSF, or a pharmacologically active mutant or derivative thereof. In still other embodiments, the hematopoietic growth factors that can be administered before, concurrently with, after, or in cycles with a vitamin D compound can be a combination of EPO and HuG-CSF, or pharmacologically active mutants or derivatives thereof. In other embodiments, the vitamin D compound can be administered as an emulsion pre-concentrate in combination with EPO, G-CSF, a pharmacologically active mutant or derivative thereof, or a combination thereof.

In certain embodiments, the range of r-HuEPO, or a pharmacologically active mutant or derivative thereof, administered to a subject to treat MDS, or ameliorate a symptom thereof, can be from about 1 Unit/kg to about 2000 Units/kg three times per week (TIW), preferably from about 10 Units/kg to about 1000 Units/kg TIW, and more preferably from about 25 Units/kg to about 500 Units/kg TIW. In other embodiments, the r-HuEPO can be administered to a subject to treat MDS, or ameliorate a symptom thereof, in a dose of about 1, 10, 20, 50, 100, 200, 300, 400, 500, 750, 1000, 1250, 1500, 1750, 2000 Units/kg or any range of doses therein. In other embodiments, the r-HuEPO can be administered in combination with one or more vitamin D compounds, wherein the one or more vitamin D compounds are administered according to the doses and schedules described herein.

In certain embodiments, the range of r-metHuG-CSF, or a pharmacologically active mutant or derivative thereof, administered to a subject to treat MDS, or ameliorate a symptom thereof, can be from about 1 μg/kg/day to about 100 μg/kg/day, preferably from about 3 μg/kg/day to about 75 μg/kg/day, and more preferably from about 5 μg/kg/day to about 50 μg/kg/day. In other embodiments, the r-metHuG-CSF can be administered to a subject to treat MDS, or ameliorate a symptom thereof, in a dose of about 1, 5, 10, 20, 30, 40, 50, 60, 70, 80, 90, 100 μg/kg or any range of doses therein. In other embodiments, the r-metHuG-CSF, or a pharmacologically active mutant or derivative thereof, can be administered in combination with one or more vitamin D compounds, wherein the one or more vitamin D compounds are administered according to the doses and schedules described herein.

In other embodiments, a vitamin D compound can be administered in combination with r-HuEPO, r-metHuG-CSF, a pharmacologically active mutant or derivative thereof, or a combination thereof, wherein r-HuEPO, r-metHuG-CSF, or their pharmacologically active mutants or derivatives are administered in the above described doses, respectively.

In certain embodiments, the hematopoietic growth factor is r-HuEPO and is administered before, concurrently with, after, or in cycles with the administration of a vitamin D compound, wherein the range of administration of r-HuEPO is from about 50 Units/kg to about 100 Units/kg TIW. In other embodiments, the hematopoietic growth factor is r-metHuG-CSF and is administered before, concurrently with, after, or in cycles with a vitamin D compound, wherein the range of administration of r-metHuG-CSF is from about 5 μg/kg/day to about 25 μg/kg/day. In yet other embodiments, the hematopoietic growth factors are a combination of r-HuEPO and r-metHuG-CSF and are administered before, concurrently with, after, or in cycles with a vitamin D compound, wherein the range of administration is from about 25 Units/kg to about 500 Units/kg TIW for r-HuEPO and from about 5 μg/kg/day to about 25 μg/kg/day for r-metHuG-CSF.

In other embodiments, the growth factor administered before, concurrently with, after, or in cycles with a vitamin D compound can be one of IL-1, IL-2, IL-3, IL-4, IL-6, IL-8, IL-11, IL-12, IFN-alpha, GM-CSF, TPO, or a pharmacologically active mutant or derivative thereof. In still other embodiments, the growth factors administered before, concurrently with, after, or in cycles with a vitamin D compound can be more than one of IL-1, IL-2, IL-3, IL-4, IL-6, IL-8, IL-11, IL-12, IFN-alpha, GM-CSF, TPO, or a pharmacologically active mutant or derivative thereof. In yet other embodiments, the vitamin D compound can be administered as an emulsion pre-concentrate in combination with one or more of r-HuEPO, r-metHuG-CSF, IL-1, IL-2, IL-3, IL-4, IL-6, IL-8, IL-11, IL-12, IFN-alpha, GM-CSF, TPO, a pharmacologically active mutant or derivative thereof, or a combination thereof.

In other embodiments, the additional active agents that are not vitamin D compounds can be immunomodulators. The immunomodulators can be administered before, concurrently with, after, or in cycles with a vitamin D compound according to the methods of the present invention. In certain embodiments, the immunomodulator can be one of ATG, ALG, thalidomide, prednisone, CyA, dexamethasone, or pentoxifylline. In other embodiments, the immunomodulators can be more than one of ATG, ALG, thalidomide, prednisone, CyA, dexamethasone, or pentoxifylline. In still other embodiments, the vitamin D compound can be administered as an emulsion pre-concentrate in combination with one or more of ATG, ALG, thalidomide, prednisone, CyA, dexamethasone, or pentoxifylline, or a combination thereof.

In a particular embodiment, the immunomodulator is ATG, wherein the range of administration of ATG is from about 10 mg/kg/day to about 100 mg/kg/day. In a preferred embodiment, the immunomodulator is ATG, wherein the range of administration of ATG is from about 35 mg/kg/day to about 45 mg/kg/day. In another specific embodiment, the immunomodulator is thalidomide, wherein the range of administration of thalidomide is from about 50 mg/day to about 500 mg/day. In a preferred embodiment, the immunomodulator is thalidomide, wherein the range of administration of thalidomide is from about 100 mg/day to about 400 mg/day.

In other embodiments, the additional active agents that are not vitamin D compounds can be cytotoxic agents. The cytotoxic agents can be administered before, concurrently with, after, or in cycles with a vitamin D compound according to the methods of the present invention. In certain embodiments, the cytotoxic agent can be an antimetabolite, an anti-microtubule agent, an alkylating agent, a platinum agent, an anthracycline, an antibiotic agent, or a topoisomerase inhibitor. In other embodiments, the cytotoxic agents can be more than one of an antimetabolite, an anti-microtubule agent, an alkylating agent, a platinum agent, an anthracycline, an antibiotic agent, or a topoisomerase inhibitor. In still other embodiments, the vitamin D compound can be administered as an emulsion pre-concentrate in combination with one or more than one of an antimetabolite, an anti-microtubule agent, an alkylating agent, a platinum agent, an anthracycline, an antibiotic agent, or a topoisomerase inhibitor, or a combination thereof.

In further embodiments, the cytotoxic agent can be one of cytarabine, melphalan, topotecan, fludarabine, etoposide, idarubicin, daunorubicin, mitoxantrone, cisplatin, paclitaxel, or cyclophosphamide. In other embodiments, the cytotoxic agents can be more than one of cytarabine, melphalan, topotecan, fludarabine, etoposide, idarubicin, daunorubicin, mitoxantrone, cisplatin, paclitaxel, or cyclophosphamide. In still other embodiments, the vitamin D compound can be administered as an emulsion pre-concentrate in combination with one or more of cytarabine, melphalan, topotecan, fludarabine, etoposide, idarubicin, daunorubicin, mitoxantrone, cisplatin, paclitaxel, or cyclophosphamide, or a combination thereof.

In a particular embodiment, the cytotoxic agent is cytarabine, wherein the range of administration of cytarabine is from about 10 mg/m²/day to about 1 g/m²/day. In a preferred embodiment, the cytotoxic agent is cytarabine, wherein the range of administration of cytarabine is from about 5 mg/m²/day to about 20 mg/m²/day. In another particular embodiment, the cytotoxic agent is idarubicin, wherein the range of administration of idarubicin is from about 9 mg/m²/day to about 18 mg/m²/day. In yet another specific embodiment, the cytotoxic agent is melphalan, wherein the range of administration of melphalan is from about 1 mg/day to about 100 mg/day. In a preferred embodiment, the cytotoxic agent is melphalan, wherein the range of administration of melphalan is from about 1 mg/day to about 5 mg/day. In still another specific embodiment, the cytotoxic agent is topotecan, wherein the range of administration of topotecan is from about 1 mg/m²/day to about 100 mg/m²/day. In a preferred embodiment, the cytotoxic agent is topotecan, wherein the range of administration of topotecan is from about 1 mg/m²/day to about 5 mg/m²/day.

In yet other embodiments, the additional active agents that are not vitamin D compounds can be one or more agents that affect RNA transcription. The agents that affect RNA transcription can be administered before, concurrently with, after, or in cycles with a vitamin D compound in accordance with the methods of the present invention. In certain embodiments, the agent that affects RNA transcription can be one of decitabine, 5-azacytidine, depsipeptides, or phenylbutyrate. In other embodiments, the agents that affect RNA transcription can be more than one of decitabine, 5-azacytidine, depsipeptides, or phenylbutyrate. In still other embodiments, the vitamin D compound can be administered as an emulsion pre-concentrate in combination with one or more of decitabine, 5-azacytidine, or depsipeptides, or a combination thereof.

In a particular embodiment, the agent that affects RNA transcription is decitabine, wherein the range of administration of decitabine is from about 10 mg/m²/day to about 200 mg/m²/day. In a preferred embodiment, the agent that affects RNA transcription is decitabine, wherein the range of administration of decitabine is from about 45 mg/m²/day to about 100 mg/m²/day. In another specific embodiment, the agent that affects RNA transcription is 5-azacytidine, wherein the range of administration of 5-azacytidine is from about 5 mg/m²/day to about 200 mg/m²/day. In a preferred embodiment, the agent that affects RNA transcription is 5-azacytidine, wherein the range of administration of 5-azacytidine is from about 10 mg/m²/day to about 75 mg/m²/day.

In other embodiments, the additional active agents that are not vitamin D compounds can be derivatives of vitamin A, E, or K. The derivatives of vitamin A, E, or K can be administered before, concurrently with, after, or in cycles with a vitamin D compound in accordance with the methods of the present invention. In certain embodiments, the derivative of vitamin A, E, or K can be one of ATRA, 13-cis-retinoic acid, tocopherol, or menatetrenone. In other embodiments, the derivatives of vitamin A, E, or K can be more than one of ATRA, 13-cis-retinoic acid, tocopherol, or menatetrenone. In still other embodiments, the vitamin D compound can be administered as an emulsion pre-concentrate in combination with one or more of ATRA, 13-cis-retinoic acid, tocopherol, or menatetrenone, or a combination thereof.

In a particular embodiment, the derivative of vitamin A, E, or K is ATRA, wherein the range of administration of ATRA is from about 10 mg/m²/day to about 200 mg/m²/day. In a preferred embodiment, the derivative of vitamin A, E, or K is ATRA, wherein the range of administration of ATRA is from about 25 mg/m²/day to about 80 mg/m²/day. In another specific embodiment, the derivative of vitamin A, E, or K is 13-cis-retinoic acid, wherein the range of administration of 13-cis-retinoic acid is from about 5 mg/m²/day to about 200 mg/m²/day. In a preferred embodiment, the derivative of vitamin A, E, or K is 13-cis-retinoic acid, wherein the range of administration of 13-cis-retinoic acid is from about 10 mg/m²/day to about 100 mg/m²/day. In still another specific embodiment, the derivative of vitamin A, E, or K is menatetrenone, wherein the range of administration of menatetrenone is from about 5 mg/day to about 200 mg/day. In a preferred embodiment, the derivative of vitamin A, E, or K is menatetrenone, wherein the range of administration of menatetrenone is from about 10 mg/day to about 100 mg/day. In yet another particular embodiment, the derivative of vitamin A, E, or K is tocopherol, wherein the range of administration of tocopherol is from about 400 IU/day to about 3000 IU/day. In a preferred embodiment, the derivative of vitamin A, E, or K is tocopherol, wherein the range of administration of tocopherol is from about 800 IU/day to about 2000 IU/day.

In other embodiments, the additional active agents that are not vitamin D compounds can be agents that specifically bind biological targets related to MDS. The agents that specifically bind biological targets related to MDS can be administered before, concurrently with, after, or in cycles with a vitamin D compound in accordance with the methods of the present invention. In certain embodiments, the agent that specifically binds biological targets related to MDS can be one of anti-VEGF, gemtuzumab ozogamicin, or TNFR:Fc. In other embodiments, the agents that specifically bind biological targets related to MDS can be more than one of anti-VEGF, gemtuzumab ozogamicin, or TNFR:Fc. In still other embodiments, the vitamin D compound can be administered as an emulsion pre-concentrate in combination with one or more of anti-VEGF, gemtuzumab ozogamicin, or TNFR:Fc, or a combination thereof. In a particular embodiment, the agent that specifically binds biological targets related to MDS is gemtuzumab ozogamicin, wherein the range of administration of gemtuzumab ozogamicin is from about 5 mg/m²/week to about 20 mg/m²/week.

In other embodiments, the additional active agents that are not vitamin D compounds can be signal transduction inhibitors. The signal transduction inhibitors can be administered before, concurrently with, after, or in cycles with a vitamin D compound in accordance with the methods of the present invention. In certain embodiments, the signal transduction inhibitors can be one or more farnesyl transferase inhibitor(s). In other embodiments, the farnesyl transferase inhibitor can be one of ZARNESTRA™ and SARASAR™. In still other embodiments, the farnesyl transferase inhibitor can be more than one of ZARNESTRA™ and SARASAR™. In yet other embodiments, the vitamin D compound can be administered as an emulsion pre-concentrate in combination with one or more of ZARNESTRA™ and SARASAR™, or a combination thereof.

In other embodiments, the signal transduction inhibitors can be tyrosine kinase inhibitors. The tyrosine kinase inhibitors can be administered before, concurrently with, after, or in cycles with a vitamin D compound in accordance with the methods of the present invention. In certain embodiments, the tyrosine kinase inhibitor can be one of SU5416, SU6668, or PTK787/ZK222584. In other embodiments, the tyrosine kinase inhibitors can be more than one of SU5416, SU6668, or PTK787/ZK222584. In still other embodiments, the vitamin D compound can be administered as an emulsion pre-concentrate in combination with one or more of SU5416, SU6668, PTK787/ZK222584, or a combination thereof.

In other embodiments, the additional active agents that are not vitamin D compounds can be aminothiols. The aminothiols can be administered before, concurrently with, after, or in cycles with a vitamin D compound in accordance with the methods of the present invention. In a specific embodiment, the aminothiol is amifostine. In another specific embodiment, the vitamin D compound can be administered as an emulsion pre-concentrate in combination with amifostine. In yet another particular embodiment, the aminothiol is amifostine, wherein the range of administration of amifostine is from about 50 mg/m²/day to about 600 mg/m²/day when administered over multiple days, or from about 600 mg/m² to about 1.2 g/m² when administered in a single dose. In a preferred embodiment, the aminothiol is amifostine, wherein the range of administration of amifostine is from about 100 mg/m²/day to about 400 mg/m²/day when administered over multiple days, or from about 740 mg/m² to about 910 mg/m² when administered in a single dose.

In yet other embodiments, the additional active agents that are not vitamin D compounds can be arsenic-containing compounds. The arsenic-containing compounds can be administered before, concurrently with, after, or in cycles with a vitamin D compound in accordance with the methods of the present invention. In a specific embodiment, the arsenic-containing compound is arsenic trioxide. In another specific embodiment, the vitamin D compound can be administered as an emulsion pre-concentrate in combination with arsenic trioxide.

The doses, dose frequencies, and durations of administration of any combination stated above may also vary according to age, body weight, response, and the past medical history of the subject as well as the route of administration, pharmacokinetic and pharmacodynamic effects of the pharmaceutical agent. These factors are routinely considered by one of skill in the art. Examples of other dosage schedules and the factors considered by one of skill in the art when designing a dosage schedule are discussed above.

### Methods of Administration

In the methods of the invention, the vitamin D compound can be administered by any method known to those of skill in the art. In certain embodiments, the vitamin D compounds can be administered by any route known to one of skill in the art that can achieve rapid onset of peak plasma concentrations of the vitamin D compounds. In other embodiments, the vitamin D compounds are administered orally, mucosally, or parenterally. For example, mucosal administration of the vitamin D compounds can include nasal, sublingual, vaginal, buccal, or rectal administration, while parenteral administration of the vitamin D compounds can include intravenous, intramuscular, or intraarterial administration. Where the vitamin D compound is administered intravenously or intraarterially, the vitamin D compound may be administered as either a bolus injection or as an infusion over several minutes to hours. In preferred embodiments, the vitamin D compounds can be administered either orally or intravenously.

The timing of the administration of the vitamin D compound can also vary. The vitamin D compound can be administered, regardless of the dosage form, as co-therapy either before, concurrently with, after, or in cycles with administration one or more additional active agent(s). The administration of the vitamin D compounds and formulations can also occur in a cycling treatment regimen such as the administration of the vitamin D between or concurrently with other treatments within a cycling series of these other treatments. In certain embodiments, the vitamin D compounds can be administered intermittently according to continuous or non-continuous periodic schedules.

Further, where the methods of the invention additionally comprise administration of one or more additional active agents, the additional active agents may be administered by any method known to one of skill in the art.

### Pharmaceutical Formulations of Vitamin D Compounds

For use in the instant invention, the pharmaceutical agent can comprise one or more vitamin D compounds or optionally one or more vitamin D compounds in combination with one or more additional active agents. The pharmaceutical agent can be administered in combination with one or more additional active agents as well, such as hematopoietic growth factors or cytokines in the treatment of MDS. The pharmaceutical agent can be in the form of any pharmaceutical formulation known to those of skill in the art. Typically, the pharmaceutical formulations and dosage forms of the present invention comprise at least one vitamin D compound or a pharmaceutically acceptable salt, solvate, hydrate, stereoisomer, clathrate, or prodrug thereof, The pharmaceutical formulations and dosage forms of the invention can further comprise one or more excipients, diluents or any other components known to persons of skill in the art and germane to the methods of formulation of the present invention. The pharmaceutical formulations and dosage forms of the present invention can further comprise other active ingredients that are not vitamin D compounds. In addition, the pharmaceutical formulations of the present invention can be used to prepare single unit dosage forms.

The composition, shape and type of dosage forms may typically vary depending on their use. For example, a dosage form used in the acute treatment of a disease may comprise larger amounts of pharmaceutical agents than a dosage form used in the chronic treatment of the same disease. Similarly, a parenteral dosage form may contain smaller amounts of pharmaceutical agents than an oral dosage form used to treat the same disease. These and other ways in which specific dosage forms may vary will be readily apparent to those skilled in the art. See, e.g., Remington's Pharmaceutical Sciences, 18th ed., Mack Publishing, Easton Pa. (1990).

The dosage forms are suitable for oral; mucosal such as nasal, sublingual, vaginal, buccal, or rectal; or parenteral such as intravenous, intramuscular, or intraarterial administration. Where the vitamin D compound is administered intravenously or intraarterially, the vitamin D compound may be administered as either a bolus injection or as an infusion over several minutes to hours. Examples of dosage forms include but are not limited to tablets, caplets, capsules such as hard and soft gelatin capsules, cachets, troches, lozenges, dispersions, suppositories, ointments, cataplasms (poultices), pastes, powders, dressings, creams, plasters, solutions, patches, aerosols such as nasal sprays or inhalers, and gels. Suitable liquid dosage forms for oral or mucosal administration include but are not limited to aqueous and non-aqueous liquid suspensions, oil-in-water emulsions, water-in-oil emulsions, solutions and elixirs. Liquid dosages include but are not limited to the reconstitution of sterile solids, which can be crystalline or amorphous, into liquid dosage forms suitable for parenteral administration.

Preferred dosage forms of the present invention include oral dosage forms and intravenous dosage forms. Where the vitamin D compounds are administered intermittently orally, the vitamin D compounds are preferably administered in the form of emulsion pre-concentrates. In a preferred embodiment of an oral dosage form of a vitamin D compound, the oral dosage form is an emulsion pre-concentrate of a vitamin D compound that comprises about 15 μg of calcitriol in addition to the following excipients with the amount given in approximate percentage by weight: 65% Miglyol 812N®, 30% Gelucire 44/14®, 5% vitamin-E TPGS and about 0.05% each of butylated hydroxytoluene (BHT) and butylated hydroxyanisole (BHA). In the most preferred embodiment of an intravenous dosage form of a vitamin D compound, the intravenous dosage form is CALCIJEX®, which can contain 1 μg calcitriol, 4 mg of Polysorbate 20, 2.5 mg of sodium ascorbate and optionally either HCl or NaOH for pH adjustment.

Typical pharmaceutical formulations and dosage forms comprise one or more excipients. Suitable excipients are well-known to those skilled in the art and non-limiting examples of suitable excipients are provided herein. Whether a particular excipient is suitable for incorporation into a pharmaceutical formulation or dosage form depends on a variety of factors well-known in the art including, but not limited to, the route by which the dosage form is administered. In one example, oral dosage forms such as tablets may contain excipients not suited for use in parenteral dosage forms. The suitability of a particular excipient may also depend on the specific pharmaceutical agent in the dosage form.

In certain embodiments, the pharmaceutical formulations and dosage forms can be anhydrous, since water, as well as heat, can facilitate the degradation of some compounds. Thus, the effect of water as well as heat on a formulation can be of great significance since moisture and/or humidity are commonly encountered during manufacture, handling, packaging, storage, shipment and use of formulations.

Anhydrous pharmaceutical formulations and dosage forms can be prepared using anhydrous or low moisture containing ingredients and low moisture or low humidity conditions. In a preferred embodiment, the anhydrous pharmaceutical formulations are prepared, stored and packaged to preserve the anhydrous environment by using materials capable of preventing exposure to water and facilitating the production of suitable formulary kits. Examples of suitable materials include but are not limited to hermetically sealed foils, plastics, and unit dose containers such as vials, blister packs and strip packs.

In certain embodiments, the pharmaceutical formulations and dosage forms comprise one or more stabilizers, which are compounds that reduce the rate at which an active ingredient will decompose and include but are not limited to antioxidants such as ascorbic acid, pH buffers, or salt buffers.

The pharmaceutical formulations of the present invention can be, for example, in a semisolid formulation or in a liquid formulation. Semisolid formulations of the present invention can be any semisolid formulation known by those of ordinary skill in the art, including, for example, gels, pastes, creams and ointments.

In certain embodiments, the pharmaceutical formulations can comprise preparations of vitamin D compounds that are currently in clinical use. Examples of such vitamin D compound preparations and analogs include but are not limited to dihydrotachysterol (DHT™, Roxane; and HYTAKEROL®, Sanofi Winthrop Pharm); calcitriol (ROCALTROL®, Roche; and CALCIJEX®, Abbott); calcifediol (CALDEROL®, Organon); ergocalciferol (CALCIFEROL®, Schwarz Pharma; and DRISDOL®, Sanofi Pharm); cholecalciferol (DELTA-D® and vitamin D₃, Freeda); paracalcitol (ZEMPLAR®, Abbott); doxercalciferol (HECTOROL®, Bone Care Int'l); and alfacalcidol (ALFAD® and ONE-ALPHA®).

ROCALTROL® is a calcitriol formulation currently in clinical use and available as capsules containing 0.25 and 0.5 μg calcitriol and as an oral solution containing 1 μg/mL calcitriol. Dosage forms of ROCALTROL® can contain additional components such as butylated hydroxyanisole (BHA) and butylated hydroxytoluene (BHT) as antioxidants. The capsules can also contain a fractionated triglyceride of coconut oil and the oral solution contains a fractionated triglyceride of palm seed oil. See Physicians' Desk Reference 2991 (56th ed., 2002).

Oral Dosage Forms

In certain embodiments, the pharmaceutical agents can be administered orally. Pharmaceutical formulations that are suitable for oral administration can be presented as discrete dosage forms including, but not limited to, tablets such as chewable tablets, caplets, capsules and liquids such as flavored syrups. Such dosage forms contain predetermined amounts of a pharmaceutical agent and may be prepared by methods of pharmacy well-known to those skilled in the art. See generally, Remington's Pharmaceutical Sciences, 18th ed., Mack Publishing, Easton Pa. (1990).

Typical oral dosage forms are prepared by combining a pharmaceutical agent with at least one excipient according to conventional pharmaceutical compounding techniques. Excipients can take a wide variety of forms. For example, excipients suitable for use in oral liquid or aerosol dosage forms include, but are not limited to, water, glycols, oils, alcohols, flavoring agents, preservatives and coloring agents. Examples of excipients suitable for use in solid oral dosage forms such as powders, tablets, capsules, and caplets include, but are not limited to, starches, sugars, micro-crystalline cellulose, diluents, granulating agents, lubricants, binders and disintegrating agents.

In certain embodiments, suitable forms of binders include, but are not limited to, corn starch, potato starch, other starches, gelatin, natural and synthetic gums such as acacia, sodium alginate, alginic acid, other alginates, powdered tragacanth, guar gum, cellulose and its derivatives. Examples of cellulose derivatives include but are not limited to ethyl cellulose, cellulose acetate, carboxymethyl cellulose calcium, sodium carboxymethyl cellulose. Other binders include but are not limited to polyvinyl pyrrolidone, methyl cellulose, pre-gelatinized starch, hydroxypropyl methyl cellulose, microcrystalline cellulose and mixtures thereof.

Suitable forms of microcrystalline cellulose include, but are not limited to, those commercially known as AVICEL®, AVICEL-PH-101®, AVICEL-PH-103®, AVICEL RC-581®, Avicel-PH-105® (Avicel® products are available from FMC Corporation, American Viscose Division, Avicel Sales, Marcus Hook, Pa.) and mixtures thereof. In certain embodiments, the binder can be a mixture of microcrystalline cellulose and sodium carboxymethyl cellulose sold as Avicel RC-581®. Suitable anhydrous or low moisture excipients or additives include AVICEL-PH-103® and STARCH 1500 LM®.

In certain embodiments, suitable fillers include, but are not limited to, talc, calcium carbonate, microcrystalline cellulose, powdered cellulose, dextrates, kaolin, mannitol, silicic acid, sorbitol, starch, pre-gelatinized starch and mixtures thereof. The binder or filler is typically present from about 50 to about 99 percent by weight of the pharmaceutical formulation or dosage form.

In certain embodiments, disintegrants may be used in the formulations to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the pharmaceutical agent should be used to form solid oral dosage forms. The amount of disintegrant varies based upon the type of formulation and is readily determinable to those of ordinary skill in the art. In certain embodiments, the pharmaceutical formulations comprise from about 0.5 to about 15 percent by weight of disintegrant. In a preferred embodiment, the pharmaceutical formulations comprise from about 1 to about 5 percent by weight of disintegrant.

In other embodiments, suitable disintegrants include but are not limited to agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums and mixtures thereof.

In certain embodiments, suitable lubricants include but are not limited to calcium stearate, magnesium stearate, mineral oil, light mineral oil, glycerin, sorbitol, mannitol, polyethylene glycol, other glycols, stearic acid, sodium lauryl sulfate, talc, and hydrogenated vegetable oils such as peanut oil, cottonseed oil, sunflower oil, sesame oil, olive oil, corn oil and soybean oil. In other embodiments, suitable lubricants include but are not limited to zinc stearate, ethyl oleate, ethyl laureate, agar and mixtures thereof. In other embodiments, suitable lubricants include but are not limited to a syloid silica gel such as Aerosil 200 (manufactured by W.R. Grace Co. of Baltimore, Md.), a coagulated aerosol of synthetic silica (marketed by Degussa Co. of Plano, TX), Cab-O-SIL® (a pyrogenic silicon dioxide product sold by Cabot Co. of Boston, Mass.) and mixtures thereof. When lubricants are used, they are typically present in an amount of less than about 1 weight percent of the pharmaceutical formulations or dosage forms.

In certain embodiments, the oral dosage form can be tablets and capsules. Tablets and capsules can comprise solid excipients and represent the most advantageous oral dosage unit forms due to their ease of delivery. Tablets can be coated by standard aqueous or nonaqueous techniques if desired. Such dosage forms can be prepared by any pharmaceutical method. In general, pharmaceutical formulations and dosage forms are prepared by uniformly and intimately admixing the pharmaceutical agents with liquid carriers, finely divided solid carriers, or both and then shaping the product into the desired form. For example, a tablet can be prepared by compression or molding. Compressed tablets can be prepared by compressing the pharmaceutical agents, and optionally excipients, in a suitable machine. Molded tablets can likewise be made by molding a mixture of the pharmaceutical agents, and optionally excipients, moistened with an inert liquid diluent in a suitable machine.

When the composition of the present invention is formulated in unit dosage form, the active vitamin D compound will preferably be present in an amount of between 1 and 200 μg per unit dose. More preferably, the amount of active vitamin D compound per unit dose will be about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 μg or any amount therein. In a preferred embodiment, the amount of active vitamin D compound per unit dose will be about 5 μg to about 180 μg, more preferably about 10 μg to about 135 μg, more preferably about 45 μg. In one embodiment, the unit dosage form comprises 45, 90, 135, or 180 μg of calcitriol.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg. In one embodiment, the total quantity is about 225, 450, 675, or 900 mg. In one embodiment, the unit dosage form is a capsule comprising 45, 90, 135, or 180 μg of calcitriol.

The relative proportion of ingredients in the formulations may vary according to the particular type of composition, the particular function of ingredients, the particular ingredients and the desired physical characteristics of the product. For example, a composition may be a free flowing liquid or a paste for topical use. Determination of workable proportions in any particular instance are within the capability of a person of ordinary skill in the art. All indicated proportions and relative weight ranges described below are indicative of preferred teachings and not intended to be limiting in any way.

Calcitriol can be light-sensitive and especially prone to oxidation. Moreover, calcitriol and other active vitamin D compounds are lipophilic, meaning that they are soluble in lipids and some organic solvents but only sparsely soluble in a polar medium such as water. As a result of the lipophilic nature of active vitamin D compounds, the dispersion of such compounds in aqueous solutions, e.g. the gastric fluids of the stomach, is significantly limited. Accordingly, the pharmacokinetic parameters of currently available active vitamin D compound formulations are suboptimal. As a result, currently available active vitamin D compound formulations tend to exhibit substantial variability of absorption in the small intestine.

However, in certain preferred embodiments of the present invention, vitamin D compounds can be formulated as emulsion pre-concentrates to improve stability, even at elevated temperatures, to improve pharmacokinetic parameters and to reduce the variability in absorption in the small intestine. Preferably, the method provides dosage forms of active vitamin D compounds, such as calcitriol, in sufficiently high concentrations to permit convenient use. The dosage form is stable at a variety of temperatures and rapidly becomes a nanodispersion in polar mediums that include but are not limited to gastric fluids, while performing within required pharmacokinetic parameters.

At high doses, the emulsion pre-concentrates exhibit a maximum blood concentration of a vitamin D compound that is at least 1.5-2 times greater than the maximum blood concentration observed with ROCALTROL®, an elimination half-life that is one half or less than the elimination half-life observed with ROCALTROL®, and a time to maximum plasma concentration that is shorter than the time to maximum plasma concentration observed with ROCALTROL®. These pharmacokinetic characteristics are beneficial in achieving high blood levels of vitamin D compounds without the onset of hypercalcemia and preferably without the onset of clinical hypercalcemia.

In certain embodiments, the emulsion pre-concentrates form an emulsion upon dilution with a polar phase component such as a liquid or solution, i.e. polar medium, that includes but is not limited to water. The ratio of polar medium to emulsion pre-concentrate is preferably 1:1 or greater. In one example, the ratio of water to composition can range from about 1:1 to about 5000:1. In another example, the ratio of water to composition can be about 1:1, 2:1, 3:1, 4:1, 5:1, 10:1, 200:1, 300:1, 500:1, 1000:1, or 5000:1, or any range of ratios therein. The skilled artisan can determine the appropriate ratio for the desired application.

In other embodiments, the emulsion can be a submicron droplet emulsion, wherein the submicron droplet emulsions possess one or more of the following characteristics: (1) spontaneous emulsion formation when the components are brought into contact despite the absence of an energy source such as heat, high shear or other substantial agitation, (2) thermodynamically stability and (3) a monophasic state.

The droplets or particles within submicron droplet emulsions may have a variety of shapes including but not limited to spheres and liquid crystals with lamellar, hexagonal or isotropic symmetries. Submicron droplet emulsions comprise droplets or particles having an average diameter ranging generally from about 50 nm to about 1000 nm, preferably from about 100 nm to about 750 nm, and more preferably from about 200 nm to about 400 nm.

In certain embodiments, the emulsion has an absorbance ranging from about 0.3 to about 15.0 at 400 nm upon dilution of the emulsion pre-concentrate with a polar medium such as water. In other embodiments, the emulsion has an absorbance ranging from about 0.3 to about 8.0 at 400 nm. In certain embodiments, the absorbance can range from about 0.4, 0.5, 0.6, 1.0, 1.2, 1.6, 2.0, 2.2, 2.4, 2.5, 3.0, or 4.0, or any range of absorbances therein at 400 nm. In a preferred embodiment, the emulsion is formed with a 100:1 dilution of water with the emulsion pre-concentrate and has an absorbance ranging from about 0.3 to about 4.0 at 400 nm. Methods for determining the absorbance of a liquid solution are well-known by those in the art. The skilled artisan will be able to ascertain and adjust the relative proportions of the ingredients of the emulsion pre-concentrates of the invention in order to obtain, upon dilution with a polar medium such as water, an emulsion having any particular absorbance encompassed within the scope of the invention.

In certain embodiments, the emulsion pre-concentrates comprise (a) one or more lipophilic phase components, (b) one or more surfactants, and (c) one or more vitamin D compounds; wherein said composition is an emulsion pre-concentrate, which upon dilution with a polar medium such as water in, for example, a water-to-composition ratio of about 1:1 or more forms an emulsion having an absorbance of greater than 0.3 at 400 nm. In certain embodiments, the emulsion pre-concentrates may further comprise either one or more hydrophobic or a hydrophilic phase components. The vitamin D compounds of the emulsion pre-concentrates are described above. The vitamin D compounds can be active vitamin D compounds or compounds that can be converted to active vitamin D compounds when administered.

The lipophilic phase components can be any pharmaceutically acceptable solvent which is not miscible with water. Typically, the lipophilic phase component comprises mono-, di- or triglycerides that include but are not limited to those derived from C₆, C₈, C₁₀, C₁₂, C₁₄, C₁₆, C₁₈, C₂₀ and C₂₂ fatty acids. Exemplary diglycerides include, in particular, diolein, dipalmitolein and mixed caprylin-caprin diglycerides. Preferred triglycerides include but are not limited to vegetable oils, fish oils, animal fats, hydrogenated vegetable oils, partially hydrogenated vegetable oils, synthetic triglycerides, modified triglycerides, fractionated triglycerides, medium and long-chain triglycerides, structured triglycerides and mixtures thereof.

Preferred triglycerides include but are not limited to almond oil, babassu oil, borage oil, blackcurrant seed oil, canola oil, castor oil, coconut oil, corn oil, cottonseed oil, evening primrose oil, grapeseed oil, groundnut oil, mustard seed oil, olive oil, palm oil, palm kernel oil, peanut oil, grapeseed oil, safflower oil, sesame oil, shark liver oil, soybean oil, sunflower oil, hydrogenated castor oil, hydrogenated coconut oil, hydrogenated palm oil, hydrogenated soybean oil, hydrogenated vegetable oil, hydrogenated cottonseed and castor oil, partially hydrogenated soybean oil, partially hydrogenated soy and cottonseed oil, glyceryl tricaproate, glyceryl tricaprylate, glyceryl tricaprate, glyceryl triundecanoate, glyceryl trilaurate, glyceryl trioleate, glyceryl trilinoleate, glyceryl trilinolenate, glyceryl tricaprylate/caprate, glyceryl tricaprylate/caprate/laurate, glyceryl tricaprylate/caprate/linoleate and glyceryl tricaprylate/caprate/stearate.

In certain embodiments, the preferred triglyceride can be a medium chain triglyceride commercially known as Labrafac CC®. Other preferred triglycerides include, for example, neutral oils such as neutral plant oils, and in particular, fractionated coconut oils such as the commercially available MIGLYOL®, which includes but is not limited to MIGLYOL 810®; MIGLYOL 812®; MIGLYOL 818®; and CAPTEX 355®. A preferred lipophilic phase component can be the product Miglyol 812®. See U.S. Pat. No. 5,342,625.

Other suitable triglycerides include but are not limited to caprylic-capric acid triglycerides such as commercially known as MYRITOL®, which includes but is not limited to MYRITOL 813®. Other suitable products of this class include but are not limited to CAPMUL MCT®, CAPTEX 200®, CAPTEX 300®, CAPTEX 800®, NEOBEE MS® and MAZOL 1400®.

As discussed above, the emulsion pre-concentrates further comprise one or more surfactants. Surfactants that can be used include but are not limited to hydrophilic or lipophilic surfactants, which include but are not limited to anionic, cationic, non-ionic and amphoteric surfactants or mixtures thereof. In preferred embodiments, surfactants are non-ionic hydrophilic and non-ionic lipophilic surfactants.

In certain embodiments, suitable hydrophilic surfactants include but are not limited to reaction products of natural or hydrogenated vegetable oils and ethylene glycol such as polyoxyethylene glycolated natural or hydrogenated vegetable or castor oils. The reaction products may be obtained by known methods which include but are not limited to the reaction of a natural or hydrogenated castor oil with ethylene oxide in a molar ratio of from about 1:35 to about 1:60. Optionally, the free polyethylene glycol components can be removed from the product using the methods taught in German Auslegeschrifien 1,182,388 and 1,518,819.

Other suitable hydrophilic surfactants include but are not limited to polyoxyethylene-sorbitan-fatty acid esters, which include but are not limited to mono-and trilauryl, palmityl, stearyl and oleyl esters such as the following commercially known TWEEN® products:

TWEEN 20® (polyoxyethylene(20)sorbitanmonolaurate),

TWEEN 40® (polyoxyethylene(20)sorbitanmonopalmitate),

TWEEN 60® (polyoxyethylene(20)sorbitanmonostearate),

TWEEN 80® (polyoxyethylene(20)sorbitanronooleate),

TWEEN 65® (polyoxyethylene(20)sorbitantristearate),

TWEEN 85® (polyoxyethylene(20)sorbitantrioleate),

TWEEN 21® (polyoxyethylene(4)sorbitanmonolaurate),

TWEEN 61® (polyoxyethylene(4)sorbitanmonostearate) and

TWEEN 81® (polyoxyethylene(5)sorbitanmonooleate).

The most preferred products of this class for use in the compositions are TWEEN 40® and TWEEN 80®. See Hauer, et al., U.S. Pat. No. 5,342,625.

In other embodiments, suitable hydrophilic surfactants include but are not limited to polyoxyethylene alkylethers, polyoxyethylene glycol fatty acid esters such as polyoxyethylene stearic acid esters, polyglycerol fatty acid esters, polyoxyethylene glycerides, polyoxyethylene vegetable oils and polyoxyethylene hydrogenated vegetable oils. Other suitable hydrophilic surfactants include but are not limited to the products of reaction mixtures of polyols and one or more members of the group consisting of fatty acids, glycerides, vegetable oils, hydrogenated vegetable oils and sterols; polyoxyethylene-polyoxypropylene co-polymers and block co-polymers; dioctylsuccinate, dioctylsodiumsulfosuccinate, di-[2-ethylhexyl]-succinate or sodium lauryl sulfate; phospholipids, and preferably lecithins such as soya bean lecithins; propylene glycol mono- and di-fatty acid esters such as, for example, propylene glycol dicaprylate, propylene glycol dilaurate, propylene glycol hydroxystearate, propylene glycol isostearate, propylene glycol laurate, propylene glycol ricinoleate and propylene glycol stearate. Most preferably, the fatty acid ester can be propylene glycol caprylic-capric acid diester. Other suitable hydrophilic surfactants include but are not limited to bile salts such as sodium taurocholate.

In certain embodiments, suitable lipophilic surfactants include but are not limited to alcohols, polyoxyethylene alkylethers, fatty acids, bile acids, glycerol fatty acid esters, acetylated glycerol fatty acid esters, lower alcohol fatty acids esters, polyethylene glycol fatty acids esters, polyethylene glycol glycerol fatty acid esters, polypropylene glycol fatty acid esters, polyoxyethylene glycerides, lactic acid esters of mono- or di-glycerides, propylene glycol diglycerides, sorbitan fatty acid esters, polyoxyethylene sorbitan fatty acid esters, polyoxyethylene-polyoxypropylene block copolymers, transesterified vegetable oils, sterols, sugar esters, sugar ethers, sucroglycerides, polyoxyethylene vegetable oils, polyoxyethylene hydrogenated vegetable oils. In other embodiments, suitable lipophilic surfactants include but are not limited to the products of reaction mixtures of polyols and one or more members of the group consisting of fatty acids, glycerides, vegetable oils, hydrogenated vegetable oils and sterols.

In other embodiments, suitable lipophilic surfactants include but are not limited to trans-esterification products of natural vegetable oil triglycerides and polyalkylene polyols. Such trans-esterification products are known in the art and may be obtained using methods taught in U.S. Pat. No. 3,288,824. These trans-esterification products include but are not limited to reaction mixtures of one or more natural vegetable oils such as maize oil, kernel oil, almond oil, ground nut oil, olive oil, palm oil with one or more polyethylene glycols. The preferred polyethylene glycols have an average molecular weight of from 200 to 800. In one preferred embodiment, the products are obtained by trans-esterification of a 2:1 molar ratio of a natural vegetable oil triglyceride to polyethylene glycol. Various forms of trans-esterification products are commercially known as LABRAFIL®.

In certain embodiments, suitable lipophilic surfactants include but are not limited to oil-soluble vitamin derivatives such as tocopherol PEG-1000 succinate (“vitamin E TPGS”), monoglycerides, diglycerides and mixtures thereof; esterification products of caprylic or capric acid with glycerol; sorbitan fatty acid esters; pentaerythritol and pentaerythrite fatty acid esters; and polyalkylene glycol ethers such as pentaerythrite-dioleate, -distearate, -monolaurate, -polyglycol ethers; monoglycerides such as glycerol monooleate, glycerol monopalmitate and glycerol monostearate; glycerol triacetate or (1,2,3)-triacetin; sterols and derivatives including but not limited to cholesterols and derivatives thereof, and in particular, phytosterols such as products comprising sitosterol, campesterol or stigmasterol; and ethylene oxide adducts such as soya sterols and derivatives thereof.

Those of ordinary skill in the art understand that several commercial surfactant compositions may contain small to moderate amounts of triglycerides. Thus, in certain embodiments, the surfactants that are suitable for use in the present pharmaceutical formulations may include surfactants containing triglycerides. Examples of commercial surfactant compositions containing triglycerides include but are not limited to GELUCIRE®, MAISINE® and IMWITOR®. Specific examples of these compounds are the saturated polyglycolized glycerides such as GELUCIRE 44/14®, GELUCIRE 50/13® and GELUCIRE 53/10®; semi-synthetic triglycerides such as GELUCIRE 33/01®, GELUCIRE 39/01®; and other GELUCIRE® surfactant compositions such as 37/06, 43/01, 35/10, 37/02, 46/07, 48/09, 50/02, 62/05, etc.

In other embodiments, suitable commercial surfactant compositions include but are not limited to linoleic glycerides such as MAISINE 35-I® and caprylic/capric glycerides such as IMWITOR 742®. See U.S. Pat. No. 6,267,985. Persons skilled in the art will recognize that there are other commercial surfactant compositions that have significant triglyceride contents, and will appreciate that compositions containing triglycerides as well as surfactants may be suitable to provide all or part of the lipophilic phase components, as well as all or part of the surfactants.

In certain embodiments, the emulsion pre-concentrates of the present invention may optionally comprise one or more hydrophilic phase components. Suitable hydrophilic phase components include but are not limited to ethers of alkanediols and preferably diethers. The one or more hydrophilic phase components may comprise, for example, a pharmaceutically acceptable C₁-C₅ alkyl or tetrahydrofurfuryl ether of a low molecular weight mono- or poly-oxy-alkanediol. The alkanediol can be a C₂-C₁₂ oxy-alkanediol and preferably a C₄ oxy-alkanediol. More preferably, the oxy-alkanediol can be straight-chained. Exemplary hydrophilic phase components for use in relation to the present invention are commercially known as TRANSCUTOL® and COLYCOFUROL®. See U.S. Pat. No. 5,342,625.

In other embodiments, the hydrophilic phase component may include one or more additional ingredients. Preferably, however, additional ingredients comprise materials in which the vitamin D compound can be sufficiently soluble, such that the performance of the hydrophilic phase component as a carrier of the vitamin D compound is not materially impaired. Other possible hydrophilic phase components include but are not limited to lower alkanols such as C₁-C₅ alkanols and preferably ethanol. In a preferred embodiment, the hydrophilic phase component comprises 1,2-propyleneglycol.

In certain embodiments, any pharmaceutical formulations of the invention, such as an emulsion pre-concentrate, may further comprise one or more additives. Additives that are well-known in the art include but are not limited to detackifiers, anti-foaming agents, buffering agents, antioxidants such as ascorbyl palmitate, butyl hydroxy anisole (BHA), butyl hydroxy toluene (BHT) and tocopherols such as α-tocopherol (vitamin E), preservatives, chelating agents, viscomodulators, tonicifiers, flavorants, colorants, odorants, opacifiers, suspending agents, binders, fillers, plasticizers, lubricants and mixtures thereof. Persons of skill in the art can readily determine the amounts of such additives required to achieve the desired properties. For example, antioxidants may be present in an amount of from about 0.05% to about 0.35% by weight based upon the total weight of the composition.

In other embodiments, the additives may also comprise thickening agents and salts thereof. Thickening agents may be included for a variety of reasons including but not limited to providing a sustained release effect. However, where oral administration is intended, thickening agents will generally not be required and are generally less preferred. Thickening agents are generally indicated for topical application. Suitable thickening agents known in the art include but are not limited to pharmaceutically acceptable polymeric materials and inorganics. In a preferred embodiment, thickening agents include but are not limited to polyacrylate and polyacrylate co-polymer resins, poly-acrylic acid and poly-acrylic acid/methacrylic acid resins, celluloses and cellulose derivatives. In certain embodiments, cellulose derivatives include but are not limited to acylated celluloses such as cellulose-acetates, cellulose-acetatephthalates, cellulose-acetatesuccinates and hydroxypropylmethyl cellulose phthalates. Salts of cellulose and cellulose derivatives include but are not limited to sodiumcarboxymethyl cellulose. Preferred cellulose derivatives include but are not limited to alkyl celluloses such as methyl, ethyl and propyl celluloses; hydroxyalkyl celluloses such as hydroxypropyl celluloses and hydroxypropylalkyl celluloses. The hydroxypropylalkyl celluloses include but are not limited to hydroxypropylmethyl celluloses.

In certain embodiments, other additives can serve as thickening agents and include but are not limited to polyvinylpyrrolidones such as poly-N-vinylpyrrolidones and vinylpyrrolidone co-polymers. Vinylpyrrolidone co-polymers include but are not limited to vinylpyrrolidone-vinylacetate co-polymers, polyvinyl resins such as polyvinylacetates and polyvinylalcohols and polymeric materials. In other embodiments, the polymeric additives include but are not limited to gum traganth, gum arabicum, alginates such as alginic acid and salts of alginic acid such as the sodium alginates. In certain embodiments, inorganic thickening agents are suitable and include but are not limited to atapulgite, bentonite and silicates. The silicates include but are not limited to hydrophilic silicon dioxide products such as alkylated silica gels and are preferably methylated. In a preferred embodiment, the inorganic thickening agents are colloidal silicon dioxide products.

In certain embodiments, the lipophilic phase components can suitably be present in an amount of from about 30% to about 90% by weight based upon the total weight of the composition. In a preferred embodiment, the lipophilic phase component can be present in an amount of from about 50% to about 85% by weight based upon the total weight of the composition.

In other embodiments, the one or more surfactants can suitably be present in an amount of from about 1% to about 50% by weight based upon the total weight of the composition. In a preferred embodiment, the one ore more surfactants can be present in an amount of from about 5% to about 40% by weight based upon the total weight of the composition. More preferably, the one or more surfactants can be present in an amount of from about 10% to about 30% by weight based upon the total weight of the composition.

The amount of vitamin D compound in compositions may vary according to a variety of factors. Examples of factors that can vary the amount of vitamin D compound include but are not limited to the intended route of administration and the extent to which other components are present. In certain embodiments, the vitamin D compound can be present in an amount of from about 0.005% to 20% by weight based upon the total weight of the composition. In other embodiments, the vitamin D compound can be present in an amount of from about 0.01% to 15% by weight based upon the total weight of the composition. In a preferred embodiment, the vitamin D compound can be present in an amount of from about 0.1% to about 10% by weight based upon the total weight of the composition.

In certain embodiments, the hydrophilic phase component can be present in an amount of from about 2% to about 20% by weight based upon the total weight of the composition. In other embodiments, the hydrophilic phase component can be present in an amount of from about 5% to 15% by weight based upon the total weight of the composition. In a preferred embodiment, the hydrophilic phase component can be present in an amount of from about 8% to 12% by weight based upon the total weight of the composition.

In certain embodiments, the emulsion pre-concentrate may be semisolid. Semisolid formulations may comprise, for example, one or more lipophilic phase components present in an amount of from about 60% to about 80% by weight based upon the total weight of the composition, one or more surfactants present in an amount of from about 5% to about 35% by weight based upon the total weight of the composition and one or more vitamin D compounds present in an amount of from about 0.01% to about 15% by weight based upon the total weight of the composition.

In certain embodiments, the emulsion pre-concentrate may be liquid. Liquid formulations may comprise, for example, one or more lipophilic phase components present in an amount of from about 50% to about 60% by weight based upon the total weight of the composition, one or more surfactants present in an amount of from about 4% to about 25% by weight based upon the total weight of the composition, one or more vitamin D compounds present in an amount of from about 0.01% to about 15% by weight based upon the total weight of the composition and one or more hydrophilic phase components present in an amount of from about 5% to about 10% by weight based upon the total weight of the composition.

Additional compositions that may be used include the following, wherein the percentage of each component is by weight based upon the total weight of the composition excluding the active vitamin D compound:

| | | |
|---|---|---|
| a. | Gelucire 44/14 | about 50% |
| | Miglyol 812 | about 50%; |
| b. | Gelucire 44/14 | about 50% |
| | Vitamin E TPGS | about 10% |
| | Miglyol 812 | about 40%; |
| c. | Gelucire 44/14 | about 50% |
| | Vitamin E TPGS | about 20% |
| | Miglyol 812 | about 30%; |
| d. | Gelucire 44/14 | about 40% |
| | Vitamin E TPGS | about 30% |
| | Miglyol 812 | about 30%; |
| e. | Gelucire 44/14 | about 40% |
| | Vitamin E TPGS | about 20% |
| | Miglyol 812 | about 40%; |
| f. | Gelucire 44/14 | about 30% |
| | Vitamin E TPGS | about 30% |
| | Miglyol 812 | about 40%; |
| g. | Gelucire 44/14 | about 20% |
| | Vitamin E TPGS | about 30% |
| | Miglyol 812 | about 50%; |
| h. | Vitamin E TPGS | about 50% |
| | Miglyol 812 | about 50%; |
| i. | Gelucire 44/14 | about 60% |
| | Vitamin E TPGS | about 25% |
| | Miglyol 812 | about 15%; |
| j. | Gelucire 50/13 | about 30% |
| | Vitamin E TPGS | about 5% |
| | Miglyol 812 | about 65%; |
| k. | Gelucire 50/13 | about 50% |
| | Miglyol 812 | about 50%; |
| l. | Gelucire 50/13 | about 50% |
| | Vitamin E TPGS | about 10% |
| | Miglyol 812 | about 40%; |
| m. | Gelucire 50/13 | about 50% |
| | Vitamin E TPGS | about 20% |
| | Miglyol 812 | about 30%; |
| n. | Gelucire 50/13 | about 40% |
| | Vitamin E TPGS | about 30% |
| | Miglyol 812 | about 30%; |
| o. | Gelucire 50/13 | about 40% |
| | Vitamin E TPGS | about 20% |
| | Miglyol 812 | about 40%; |
| p. | Gelucire 50/13 | about 30% |
| | Vitamin E TPGS | about 30% |
| | Miglyol 812 | about 40%; |
| q. | Gelucire 50/13 | about 20% |
| | Vitamin E TPGS | about 30% |
| | Miglyol 812 | about 50%; |
| r. | Gelucire 50/13 | about 60% |
| | Vitamin E TPGS | about 25% |
| | Miglyol 812 | about 15%; |
| s. | Gelucire 44/14 | about 50% |
| | PEG 4000 | about 50%; |
| t. | Gelucire 50/13 | about 50% |
| | PEG 4000 | about 50%; |
| u. | Vitamin E TPGS | about 50% |
| | PEG 4000 | about 50%; |
| v. | Gelucire 44/14 | about 33.3% |
| | Vitamin E TPGS | about 33.3% |
| | PEG 4000 | about 33.3%; |
| w. | Gelucire 50/13 | about 33.3% |
| | Vitamin E TPGS | about 33.3% |
| | PEG 4000 | about 33.3%; |
| x. | Gelucire 44/14 | about 50% |
| | Vitamin E TPGS | about 50%; |
| y. | Gelucire 50/13 | about 50% |
| | Vitamin E TPGS | about 50%; |
| z. | Vitamin E TPGS | about 5% |
| | Miglyol 812 | about 95%; |
| aa. | Vitamin E TPGS | about 5% |
| | Miglyol 812 | about 65% |
| | PEG 4000 | about 30%; |
| ab. | Vitamin E TPGS | about 10% |
| | Miglyol 812 | about 90%; |
| ac. | Vitamin E TPGS | about 5% |
| | Miglyol 812 | about 85% |
| | PEG 4000 | about 10%; and |
| ad. | Vitamin E TPGS | about 10% |
| | Miglyol 812 | about 80% |
| | PEG 4000 | about 10%. |

In one embodiment of the invention, the pharmaceutical compositions comprise an active vitamin D compound, a lipophilic component, and a surfactant. The lipophilic component may be present in any percentage from about 1% to about 100%. The lipophilic component may be present at about 1,2,3,4,5,6,7,8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The surfactant may be present in any percentage from about 1% to about 100%. The surfactant may be present at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In one embodiment, the lipophilic component is MIGLYOL 812 and the surfactant is vitamin E TPGS. In preferred embodiments, the pharmaceutical compositions comprise 50% MIGLYOL 812 and 50% vitamin E TPGS, 90% MIGLYOL 812 and 10% vitamin E TPGS, or 95% MIGLYOL 812 and 5% vitamin E TPGS.

In another embodiment of the invention, the pharmaceutical compositions comprise an active vitamin D compound and a lipophilic component, e.g., around 100% MIGLYOL 812.

In a preferred embodiment, the pharmaceutical compositions comprise 50% MIGLYOL 812, 50% vitamin E TPGS, and small amounts of BHA and BHT. This formulation has been shown to be unexpectedly stable, both chemically and physically (see Example 3). The enhanced stability provides the compositions with a longer shelf life. Importantly, the stability also allows the compositions to be stored at room temperature, thereby avoiding the complication and cost of storage under refrigeration. Additionally, this composition is suitable for oral administration and has been shown to be capable of solubilizing high doses of active vitamin D compound, thereby enabling high dose pulse administration of active vitamin D compounds for the treatment of hyperproliferative diseases and other disorders.

Parenteral Dosage Forms

In certain embodiments, the pharmaceutical agents can be administered parenterally. Parenteral dosage forms can be administered by various routes including but not limited to intravenous, including but not limited to bolus and drip injections, intramuscular and intraarterial. In preferred embodiments, the parenteral dosage forms are sterile or capable of being sterilized prior to administration to a subject since they typically bypass the subject's natural defenses against contaminants. Examples of parenteral dosage forms include but are not limited to solutions ready for injection, dry products ready to be dissolved or suspended in a pharmaceutically acceptable vehicle for injection, suspensions ready for injection and emulsions. The formulations useful in the present invention include but are not limited to CALCIJEX®, which is an example of a currently available intravenous vitamin D compound formulation which can contain 1 μg calcitriol, 4 mg of Polysorbate 20, 2.5 mg of sodium ascorbate and optionally either HCl or NaOH for pH adjustment.

Suitable vehicles that can be used to provide parenteral dosage forms of the invention are well-known to those skilled in the art. In certain embodiments, suitable vehicles for parenteral dosage forms include but are not limited to Water for Injection USP; aqueous vehicles including but not limited to Sodium Chloride Injection, Ringer's Injection, Dextrose Injection, Dextrose and Sodium Chloride Injection and Lactated Ringer's Injection; water-miscible vehicles including but not limited to ethyl alcohol, polyethylene glycol and polypropylene glycol; and non-aqueous vehicles including but not limited to corn oil, cottonseed oil, peanut oil, sesame oil, ethyl oleate, isopropyl myristate and benzyl benzoate.

In other embodiments, compounds that increase the solubility of the pharmaceutical agents can be incorporated into the parenteral dosage forms. For example, cyclodextrin and its derivatives can be used to increase the solubility of a thalidomide analogue and its derivatives. See, e.g., U.S. Pat. No. 5,134,127, which is incorporated herein by reference.

### Articles of Manufacture

The present invention also encompasses a finished packaged and labeled pharmaceutical product. This article of manufacture includes the appropriate unit dosage form in an appropriate vessel or container such as a glass vial or other container that is hermetically sealed. In the case of dosage forms suitable for parenteral administration, the active ingredient, e.g. one or more vitamin D compounds which are preferably active and more preferably calcitriol, is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral delivery.

In certain embodiments, the unit dosage form is suitable for intravenous delivery. Thus, the invention also encompasses solutions, which are preferably sterile, suitable for intravenous delivery. In an preferred embodiment, the dosage form is a solution suitable for intravenous administration, comprising at least one unit dosage form of one or more vitamin D compounds, such as, e.g., CALCIJEX®.

In an equally preferred embodiment, the unit dosage form is an oral emulsion pre-concentrate and comprises about 15 μg of calcitriol in addition to the following excipients with the amount given in approximate percentage by weight: 65% MIGLYOL 812N®, 30% GELUCIRE 44/14®, 5% vitamin-E TPGS and about 0.05% each of butylated hydroxytoluene (BHT) and butylated hydroxyanisole (BHA).

As with any pharmaceutical product, the packaging material and container are designed to protect the stability of the product during storage and shipment. For example, the packaging material and container can be designed to protect the product from light and from high temperatures in order to protect the stability of the product. Further, the products of the invention include instructions for use or other informational material that advise the physician, technician or patient on how to appropriately treat the disease or disorder in question. In other words, the article of manufacture includes instruction means indicating or suggesting a dosing regimen including but not limited to actual doses and monitoring procedures.

Specifically, the invention provides an article of manufacture comprising packaging material such as a box, bottle, tube, vial, container, intravenous (i.v.) bag, envelope and the like; and at least one unit dosage form of a pharmaceutical agent contained within said packaging material, wherein said pharmaceutical agent comprises one or more vitamin D compounds, which are preferably active and more preferably calcitriol, and said packaging material includes instruction means which indicate that said vitamin D compound can be used to treat MDS, or ameliorate a symptom thereof, by administering the specific doses and using the specific dosing regimens described herein. More specifically, the invention provides an article of manufacture comprising packaging material such as a box, bottle, tube, vial, container, intravenous (i.v.) bag, envelope and the like; and at least one unit dosage form of a pharmaceutical agent contained within said packaging material, wherein said pharmaceutical agent comprises one or more vitamin D compounds, which are preferably active and more preferably calcitriol, and wherein said packaging material includes instruction means which indicate that said vitamin D compound can be used to treat MDS by administering specific doses and using specific dosing regimens as described herein.

The invention provides an article of manufacture comprising packaging material, such as a box, bottle, tube, vial, container, intravenous (i.v.) bag, envelope and the like; and at least one unit dosage form of each pharmaceutical agent contained within said packaging material, wherein one pharmaceutical agent comprises one or more vitamin D compounds, which are preferably active and more preferably calcitriol, and the other pharmaceutical agent comprises a therapeutic agent other than a vitamin D compound, and wherein said packaging material includes instruction means which indicate that said agents can be used to treat the symptoms of MDS by administering the specific doses and using the specific dosing regimens as described herein. More specifically, the invention provides an article of manufacture comprising packaging material, such as a box, bottle, tube, vial, container, intravenous (i.v.) bag, envelope and the like; and at least one unit dosage form of each pharmaceutical agent contained within said packaging material, wherein one pharmaceutical agent comprises one or more vitamin D compounds and the other pharmaceutical agent comprises a therapeutic agent other than a vitamin D compound, and wherein said packaging material includes instruction means which indicate that said agents can be used to treat MDS by administering specific doses and using specific dosing regimens as described herein.

The invention provides an article of manufacture comprising packaging material, such as a box, bottle, tube, vial, container, intravenous (i.v.) bag, envelope and the like; and at least one unit dosage form of each pharmaceutical agent contained within said packaging material, wherein one pharmaceutical agent comprises one or more vitamin D compounds, which are preferably active and more preferably calcitriol, and the other pharmaceutical agent comprises one or more hematopoietic growth factors or cytokines, which are preferably r-HuEPO, r-metHuG-CSF or any combination thereof, and wherein said packaging material includes instruction means which indicate that said agents can be used to treat the symptoms of MDS by administering specific doses and using specific dosing regimens as described herein. More specifically, the invention provides an article of manufacture comprising packaging material, such as a box, bottle, tube, vial, container, intravenous (i.v.) bag, envelope and the like; and at least one unit dosage form of each pharmaceutical agent contained within said packaging material, wherein one pharmaceutical agent comprises one or more vitamin D compounds, which are preferably active and more preferably calcitriol, and the other pharmaceutical agent comprises one or more hematopoietic growth factors or cytokines, which are preferably r-HuEPO, r-metHuG-CSF or any combination thereof, and wherein said packaging material includes instruction means which indicate that said agents can be used to treat MDS by administering specific doses and using specific dosing regimens as described herein.

In a preferred embodiment, the instruction means enclosed in an article of manufacture indicates or suggests that the plasma concentration of calcium be monitored one or more times before and/or after a dose. For example, the instruction means enclosed in an article of manufacture can indicate that the blood calcium concentration be taken before the first dose and after one or more subsequent doses. In a specific embodiment, the instruction means enclosed in an article of manufacture indicates that the vitamin D compounds are used to treat MDS in a subject, and that the blood calcium concentration in said subject is less than about 10.5 mg/dL. In another specific embodiment, the instruction means enclosed in an article of manufacture indicates that the vitamin D compounds are used to treat the anemia of MDS, and that the blood calcium concentration in said subject is less than about 10.5 mg/dL.

The informational material enclosed in an article of manufacture for use in treating MDS, or ameliorating one or more symptoms thereof, also indicates that subjects with hypercalcemia are not administered a pharmaceutical composition comprising a vitamin D compound. In a specific embodiment, the informational material enclosed in an article of manufacture for use in treating MDS, or ameliorating one or more symptoms thereof, also indicates that subjects with grade 2, grade 3 or grade 4 of hypercalcemia are not administered a pharmaceutical composition comprising a vitamin D compound.

### EXAMPLES

### Example 1

### Pharmacokinetics of Calcitriol Administration

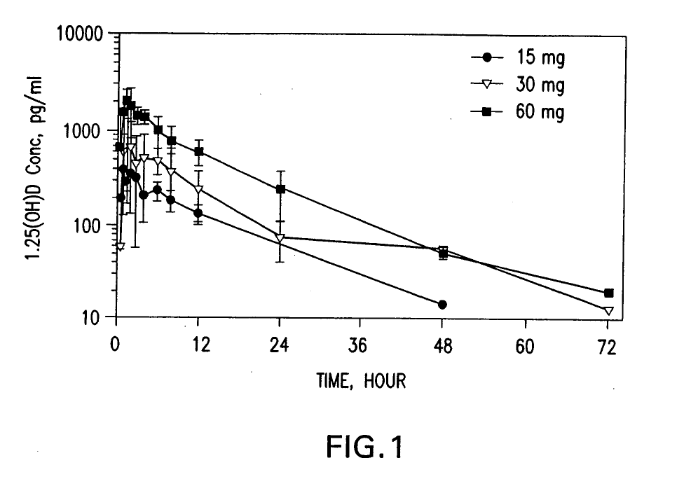

Twelve human subjects received various amounts of calcitriol in a study designed to determine the pharmacokinetic behavior of the preferred calcitriol oral dosage form. The preferred oral dosage form (“the preferred formulation”) comprises about 15 μg of calcitriol in addition to the following excipients with the amount given in approximate percentage by weight: 65% MIGLYOL 812N®, 30% GELUCIRE 44/14®, 5% vitamin-E TPGS and about 0.05% each of butylated hydroxytoluene (BHT) and butylated hydroxyanisole (BHA). Three of the subjects received 15 μg, three received 30 μg, and six received 60 μg of the preferred formulation. Blood samples were obtained pre-dose and at 0.5, 1.0, 1.5, 2.0, 3.0, 4.0, 6.0, 8.0, 12.0, 24.0, 48.0 and 72.0 hours after the initial dose (“post-dose”) of the preferred formulation. Calcitriol levels were analyzed using a commercial radioimmunoassay. Mean plasma concentration vs. time curves were plotted for each group and are shown in FIG. 1.

Non-compartmental pharmacokinetic parameters were calculated for each subject and then averaged and tabulated in Table 1. Baseline calcitriol values were subtracted from the post-dose values to adjust for endogenous calcitriol. The pharmacokinetic parameters calculated were maximal concentration in plasma (“C_{MAX}”), time at maximal concentration (“t_{MAX}”), half-life (“t_{1/2}”), and trapezoidal area determined from the concentration vs. time data from time 0 to 24 hours (“AUC₀₋₂₄”), from time 0-72 hours (“AUC₀₋₇₂”) and from time 0 to infinity (“AUC0_{-∞}”).

TABLE 1
Pharmacokinetic parameters for the preferred formulation of calcitriol as
administered to human subjects in the amounts of 15, 30 and 60 μg.

| | Dose Group | | | | | |
|---|---|---|---|---|---|---|
| Parameter | 15 μg | | 30 μg | | 60 μg | |
| C_{MAX}, pg/mL (±SD) | 398.4 | (12.9) | 898.8 | (333.6) | 1738.6 | (347.2) |
| t_{MAX}, hours | 1.00 | (1.00—1.00) | 1.50 | (1.50-2.00) | 4.00 | (1.50-4.00) |
| (median and range) | | | | | | |
| AUC-_{0-24 h}, | 3665.7 | (NA) | 6955.9 | (2825.4) | 17480.6 | (2989.7) |
| pg h/mL (±SD) | | | | | | |
| AUC-_{0-48 h}, | 5627.3 | (637.1) | 9792.9 | (2323.9) | 20999.4 | (4762.5) |
| pg h/mL (±SD) | | | | | | |
| AUC_{0-∞}, | 5464.8 | (892.8) | 11069.7 | (1406.4) | 21795.0 | (5124.8) |
| pg h/mL (±SD) | | | | | | |

| | | | |
|---|---|---|---|
| t_{1/2}, hours, | 8.9 | 16.3 | 7.3 |
| (harmonic mean, | | | |
| based on jackknife | | | |
| variance) | | | |

The pharmacokinetic data show that the preferred formulation responds linearly and predictably to increasing dosages and there was no evidence of saturation of absorption. Further, the pharmacokinetic data show that the administered doses of calcitriol achieve higher peak plasma concentrations than previously had been believed to be possible without inducing hypercalcemia. Thus, the methods of the invention provide a safe and effective method for achieving high peak plasma concentrations of vitamin D compounds to treat MDS, or ameliorate a symptom thereof, without causing hypercalcemia.

### Example 2

### Vitamin D Monotherapy

The following treatment program provides an example of use of the above-described methods to treat MDS, or ameliorate a symptom thereof.

Subjects self-administer the preferred formulation, which comprises about 15 μg of calcitriol, in addition to the following excipients with the amounts given in approximate percentage by weight: 65% MIGLYOL 812N®, 30% GELUCIRE 44/14®, 5% vitamin-E TPGS, and about 0.05% each of butylated hydroxytoluene (BHT) and butylated hydroxyanisole (BHA). The total dose administered is 45 μg of calcitriol, or three 15 μg capsules, once per week, taken all at once. The subjects are monitored every other week, and the frequency of administration or dosage of calcitriol may be modified accordingly during the duration of treatment.

Monitoring of the subjects comprises physical examination, ECOG performance status, hematology, anemia work-up, hematology, blood chemistry, urinalysis, study drug administration, transfusion record, adverse events, concomitant medications, FACT-An questionnaire, bone marrow aspirate and biopsy, peripheral blood smear, endogenous EPO, and iron status.

### Example 3

### Vitamin D Combination Treatment Program

The following treatment program provides another example of use of the above-described methods to treat MDS, or ameliorate a symptom thereof.

Subjects self-administer the preferred formulation, which comprises about 15 μg of calcitriol, in addition to the following excipients with the amounts given in approximate percentage by weight: 65% Miglyol 812N®, 30% Gelucire 44/14®, 5% vitamin-E TPGS, and about 0.05% each of butylated hydroxytoluene (BHT) and butylated hydroxyanisole (BHA). The total dose administered is 45 μg of calcitriol, or three 15 μg capsules, once per week, taken all at once. The subjects are monitored every other week, and the frequency of administration or dosage of calcitriol may be modified accordingly during the duration of treatment.

Monitoring of the subjects comprises physical examination, ECOG performance status, hematology, anemia work-up, hematology, blood chemistry, urinalysis, study drug administration, transfusion record, adverse events, concomitant medications, FACT-An questionnaire, bone marrow aspirate and biopsy, peripheral blood smear, endogenous EPO, and iron status. The subjects are further monitored to determine whether they are erythroid responders or non-responders to the vitamin D compound administration. A major erythroid responder is a subject with a baseline hemoglobin of less than 11 g/dL who experiences an increase of greater than or equal to 2 g/dL from the baseline. A minor erythroid responder is a subject with a baseline hemoglobin of less than 11 g/dL who experiences an increase of 1 to 2 g/dL from baseline. Responders continue to receive calcitriol at the same dose and frequency, while non-responders begin taking EPO in combination with the vitamin D compounds. The starting dose of EPO is 10,000 U once per day. If there is no improvement after six weeks, the dose of EPO is increased to 20,000 U once per day. EPO is administered subcutaneously, and the subject's iron status is also monitored.

### Example 4

### Clinical Trials

Patients having low risk MDS and refractory anemia unresponsive to erythropoietin were entered into a Phase 2 trial to evaluate the effect of high dose pulse administration of calcitriol. These patients are red blood cell transfusion dependent because of severe anemia. Patients were administered weekly oral calcitriol at a dose of 45 μg for 20 consecutive weeks. The calcitriol was formulated in a composition containing the following excipients with the amount given in approximate percentage by weight: 65% MIGLYOL 812N®, 30% GELUCIRE 44/14®, 5% vitamin-E TPGS and about 0.05% each of butylated hydroxytoluene (BHT) and butylated hydroxyanisole (BHA). Patients were monitored for the activity of the calcitriol by measuring hemoglobin and hematocrit levels and the number of transfusions administered during the treatment period as compared to comparable values obtained during the eight week pretreatment observation period.

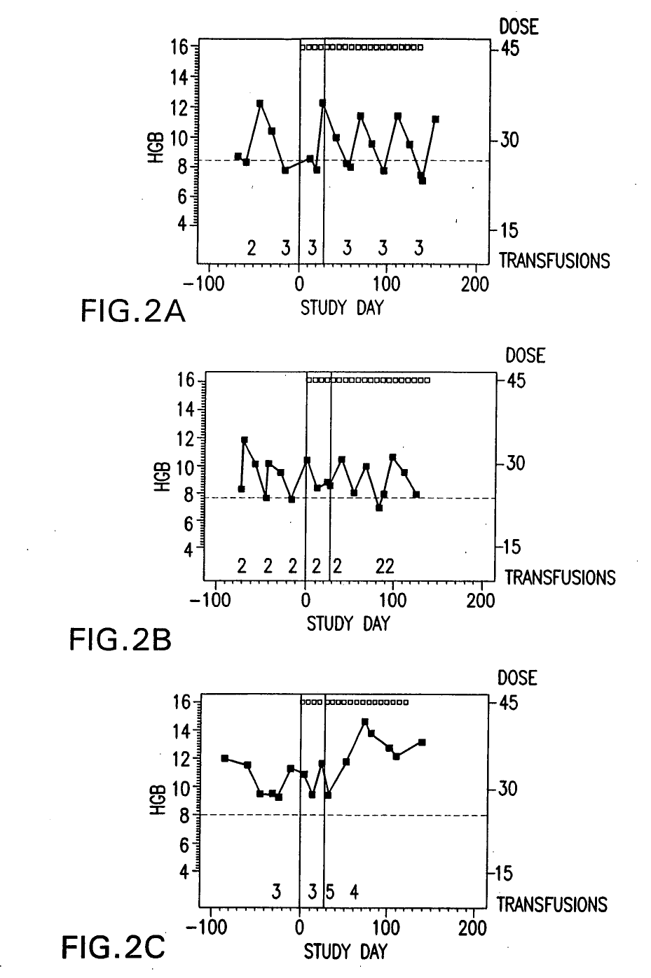

Patient #1 exhibited a rise in hemoglobin of more than one gram per deciliter as compared to baseline, constituting a protocol defined “minor response” (FIG. 2A). Patient #2 demonstrated a 50% decrease in required red blood cell transfusions as compared to baseline (FIG. 2B). Patient #3 exhibited a rise in hemoglobin of more than two grams per deciliter as compared to baseline, constituting a protocol defined “major response” (FIG. 2C). The results from all three patients are indicative of a beneficial effect of high dose pulse administration of calcitriol for the treatment of MDS.

### Example 5

### Stable Unit Dose Formulations

Formulations of calcitriol were prepared to yield the compositions in Table 2. The Vitamin E TPGS was warmed to approximately 50° C. and mixed in the appropriate ratio with MIGLYOL 812. BHA and BHT were added to each formulation to achieve 0.35% w/w of each in the final preparations.

TABLE 2
Calcitriol formulations

| | MIGLYOL | Vitamin E TPGS |
|---|---|---|
| Formulation # | (% wt/wt) | (% wt/wt) |
| 1 | 100 | 0 |
| 2 | 95 | 5 |
| 3 | 90 | 10 |
| 4 | 50 | 50 |

After formulation preparation, Formulations 2-4 were heated to approximately 50° C. and mixed with calcitriol to produce 0.1 μg calcitriol/mg total formulation. The formulations contained calcitriol were then added (˜250 μL) to a 25 mL volumetric flask and deionized water was added to the 25 mL mark. The solutions were then vortexed and the absorbance of each formulation was measured at 400 nm immediately after mixing (initial) and up to 10 min after mixing. As shown in Table 3, all three formulations produced an opalescent solution upon mixing with water. Formulation 4 appeared to form a stable suspension with no observable change in absorbance at 400 nm after 10 min.

TABLE 3
Absorption of formulations suspended in water

Absorbance at 400 nm

| Formulation # | Initial | 10 min |
|---|---|---|
| 2 | 0.7705 | 0.6010 |
| 3 | 1.2312 | 1.1560 |
| 4 | 3.1265 | 3.1265 |

To further assess the formulations of calcitriol, a solubility study was conducted to evaluate the amount of calcitriol soluble in each formulation. Calcitriol concentrations from 0.1 to 0.6 μg calcitriol/mg formulation were prepared by heating the formulations to 50° C. followed by addition of the appropriate mass of calcitriol. The formulations were then allowed to cool to room temperature and the presence of undissolved calcitriol was determined by a light microscope with and without polarizing light. For each formulation, calcitriol was soluble at the highest concentration tested, 0.6 μg calcitriol/mg formulation.

A 45 μg calcitriol dose is currently being used in Phase 2 human clinical trials. To develop a capsule with this dosage each formulation was prepared with 0.2 μg calcitriol/mg formulation and 0.35% w/w of both BHA and BHT. The bulk formulation mixtures were filled into Size 3 hard gelatin capsules at a mass of 225 mg (45 μg calcitriol). The capsules were then analyzed for stability at 5° C., 25° C./60% relative humidity (RH), 30° C./65% RH, and 40° C./75% RH. At the appropriate time points, the stability samples were analyzed for content of intact calcitriol and dissolution of the capsules. The calcitriol content of the capsules was determined by dissolving three opened capsules in 5 mL of methanol and held at 5° C. prior to analysis. The dissolved samples were then analyzed by reversed phase HPLC. A Phemonex Hypersil BDS C18 column at 30° C. was used with a gradient of acetonitrile from 55% acetonitrile in water to 95% acetonitrile at a flow rate of 1.0 mL/min during elution. Peaks were detected at 265 nm and a 25 μL sample was injected for each run. The peak area of the sample was compared to a reference standard to calculate the calcitriol content as reported in Table 4. The dissolution test was performed by placing one capsule in each of six low volume dissolution containers with 50 mL of deionized water containing 0.5% sodium dodecyl sulfate. Samples were taken at 30, 60 and 90 min after mixing at 75 rpm and 37° C. Calcitriol content of the samples was determined by injection of 100 μL samples onto a Betasil C18 column operated at 1 mL/min with a mobile phase of 50:40:10 acetonitrile:water:tetrahydrofuran at 30° C. (peak detection at 265 nm). The mean value from the 90 min dissolution test results of the six capsules was reported (Table 5).

TABLE 4
Chemical stability of calcitriol formulation in hard gelatin capsules
(225 mg total mass filled per capsule, 45 μg calcitriol)

| Storage | Time | Assay^{a} (%) | | | |
|---|---|---|---|---|---|
| Condition | (mos) | Form. 1 | Form. 2 | Form 3 | Form 4 |
| N/A | 0 | 100.1 | 98.8 | 99.1 | 100.3 |
| 5° C. | 1.0 | 99.4 | 98.9 | 98.9 | 104.3 |
| 25° C./60% RH | 0.5 | 99.4 | 97.7 | 97.8 | 102.3 |
| | 1.0 | 97.1 | 95.8 | 97.8 | 100.3 |
| | 3.0 | 95.2 | 93.6 | 96.8 | 97.9 |
| 30° C./65% RH | 0.5 | 98.7 | 97.7 | 96.8 | 100.7 |
| | 1.0 | 95.8 | 96.3 | 97.3 | 100.4 |
| | 3.0 | 94.2 | 93.6 | 95.5 | 93.4 |
| 40° C./75% RH | 0.5 | 96.4 | 96.7 | 98.2 | 97.1 |
| | 1.0 | 96.1 | 98.6 | 98.5 | 99.3 |
| | 3.0 | 92.3 | 92.4 | 93.0 | 96.4 |

^{a}Assay results indicate % of calcitriol relative to expected value based upon 45 μg content per capsule. Values include pre-calcitriol which is an active isomer of calcitriol.

TABLE 5
Physical Stability of Calcitriol Formulation in Hard Gelatin Capsules
(225 mg total mass filled per capsule, 45 μg calcitriol)

| Storage | Time | Dissolution^{a} (%) | | | |
|---|---|---|---|---|---|
| Condition | (mos) | Form. 1 | Form. 2 | Form 3 | Form 4 |
| N/A | 0 | 70.5 | 93.9 | 92.1 | 100.1 |
| 5° C. | 1.0 | 71.0 | 92.3 | 96.0 | 100.4 |
| 25° C./60% RH | 0.5 | 65.0 | 89.0 | 90.1 | 98.3 |
| | 1.0 | 66.1 | 90.8 | 94.5 | 96.2 |
| | 3.0 | 64.3 | 85.5 | 90.0 | 91.4 |
| 30° C./65% RH | 0.5 | 62.1 | 88.8 | 91.5 | 97.9 |
| | 1.0 | 65.1 | 89.4 | 95.5 | 98.1 |
| | 3.0 | 57.7 | 86.4 | 89.5 | 88.8 |
| 40° C./75% RH | 0.5 | 91.9 | 90.2 | 92.9 | 93.1 |
| | 1.0 | 63.4 | 93.8 | 94.5 | 95.2 |
| | 3.0 | 59.3 | 83.6 | 87.4 | 91.1 |

^{a}Dissolution of capsules was performed as described and the % calcitriol is calculated based upon a standard and the expected content of 45 μg calcitriol per capsule. The active isomer, pre-calcitriol, is not included in the calculation of % calcitriol dissolved. Values reported are from the 90 min sample.

The chemical stability results indicated that decreasing the MIGLYOL 812 content with a concomitant increase in Vitamin E TPGS content provided enhanced recovery of intact calcitriol as noted in Table 4. Formulation 4 (50:50 MIGLYOL 812/Vitamin E TPGS) was the most chemically stable formulation with only minor decreases in recovery of intact calcitriol after 3 months at 25° C./60% RH, enabling room temperature storage.

The physical stability of the formulations was assessed by the dissolution behavior of the capsules after storage at each stability condition. As with the chemical stability, decreasing the MIGLYOL 812 content and increasing the Vitamin E TPGS content improved the dissolution properties of the formulation (Table 5). Formulation 4 (50:50 MIGLYOL 812/Vitamin E TPGS) had the best dissolution properties with suitable stability for room temperature storage.

Various embodiments of the invention have been described. The descriptions and examples are intended to be illustrative of the invention and not limiting. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

1. A method of treating myelodysplastic syndrome (MDS), comprising administering a high dose of one or more vitamin D compounds to a subject in need thereof.

2-36. (canceled)
